(19) United States
(12) Patent Application Publication
(10) Pub. No.: US 2003/0114448 A1
(43) Pub. Date: Jun. 19, 2003

(54) Inhibitors of factor Xa

(75) Inventors: Penglie Zhang, Foster City, CA (US); Bing-Yan Zhu, Palo Alto, CA (US); Wenrong Huang, Cupertino, CA (US); Robert M. Scarborough, Half Moon Bay, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Millennium Pharmaceuticals, Inc., Cambridge, MA

(21) Appl. No.: 10/171,804

(22) Filed: May 28, 2002

Related U.S. Application Data
(60) Provisional application No. 60/294,273, filed on May 31, 2001.

(51) Int. Cl. A61K 31/541; A61K 31/5377; A61K 31/454; A61K 31/496; C07D 417/02; C07D 413/02; C 07D 4 3/02

(52) U.S. Cl. 514/228.2; 514/233.5; 514/254.04; 514/254.08; 514/254.06; 514/321; 514/323; 514/373; 514/405; 514/416; 544/60; 544/135; 544/137; 544/144; 544/371; 544/373; 544/368; 546/198; 546/199; 546/200

(57) ABSTRACT

Novel compounds, their salts and compositions related thereto having activity against mammalian factor Xa are disclosed. The compounds are useful in vitro or in vivo for preventing or treating coagulation disorders.

### CROSS-REFERENCES TO RELATED APPLICATIONS

This application claims the benefit of provisional application Ser. No. 60/294,273, filed May 31, 2001, the contents of which are incorporated herein by reference.

### STATEMENT AS TO RIGHTS TO INVENTIONS MADE UNDER FEDERALLY SPONSORED RESEARCH OR DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK.

Not Applicable

### BACKGROUND OF THE INVENTION

Field of the Invention

This invention relates to novel compounds which are potent and highly selective inhibitors of isolated factor Xa or when assembled in the prothrombinase complex. These compounds show selectivity for factor Xa versus other proteases of the coagulation (e.g. thrombin, fvIIa, fIXa) or the fibrinolytic cascades (e.g. plasminogen activators, plasmin). In another aspect, the present invention relates to novel monoamidino-containing compounds, their pharmaceutically acceptable salts, and pharmaceutically acceptable compositions thereof which are useful as potent and specific inhibitors of blood coagulation in mammals. In yet another aspect, the invention relates to methods for using these inhibitors as therapeutic agents for disease states in mammals characterized by coagulation disorders.

Hemostasis, the control of bleeding, occurs by surgical means, or by the physiological properties of vasoconstriction and coagulation. This invention is particularly concerned with blood coagulation and ways in which it assists in maintaining the integrity of mammalian circulation after injury, inflammation, disease, congenital defect, dysfunction or other disruption. Although platelets and blood coagulation are both involved in thrombus formation, certain components of the coagulation cascade are primarily responsible for the amplification or acceleration of the processes involved in platelet aggregation and fibrin deposition.

Thrombin is a key enzyme in the coagulation cascade as well as in hemostasis. Thrombin plays a central role in thrombosis through its ability to catalyze the conversion of fibrinogen into fibrin and through its potent platelet activation activity. Direct or indirect inhibition of thrombin activity has been the focus of a variety of recent anticoagulant strategies as reviewed by Claeson, G., “Synthetic Peptides and Peptidomimetics as Substrates and Inhibitors of Thrombin and Other Proteases in the Blood Coagulation System”, Blood Coag. Fibrinol., 5:411-436 (1994). Several classes of anticoagulants currently used in the clinic directly or indirectly affect thrombin (i.e. heparins, low-molecular weight heparins, heparin-like compounds and coumarins).

A prothrombinase complex, including Factor Xa (a serine protease, the activated form of its Factor X precursor and a member of the calcium ion binding, gamma carboxyglutamyl (Gla)-containing, vitamin K dependent, blood coagulation glycoprotein family), converts the zymogen prothrombin into the active procoagulant thrombin. Unlike thrombin, which acts on a variety of protein substrates as well as at a specific receptor, factor Xa appears to have a single physiologic substrate, namely prothrombin. Since one molecule of factor Xa may be able to generate up to 138 molecules of thrombin (Elodi et al., Thromb. Res. 15:617-619 (1979)), direct inhibition of factor Xa as a way of indirectly inhibiting the formation of thrombin may be an efficient anticoagulant strategy. Therefore, it has been suggested that compounds which selectively inhibit factor Xa may be useful as in vitro diagnostic agents, or for therapeutic administration in certain thrombotic disorders, see e.g., WO 94/13693.

Polypeptides derived from hematophagous organisms have been reported which are highly potent and specific inhibitors of factor Xa. U.S. Pat. No. 4,588,587 describes anticoagulant activity in the saliva of the Mexican leech, Haementeria officinalis. A principal component of this saliva was shown to be the polypeptide factor Xa inhibitor, antistasin (ATS), by Nutt, E. et al., “The Amino Acid Sequence of Antistasin, a Potent Inhibitor of Factor Xa Reveals a Repeated Internal Structure”, J. Biol. Chem., 263:10162-10167 (1988). Another potent and highly specific inhibitor of Factor Xa, called tick anticoagulant peptide (TAP), has been isolated from the whole body extract of the soft tick Ornithidoros moubata, as reported by Waxman, L., et al., “Tick Anticoagulant Peptide (TAP) is a Novel Inhibitor of Blood Coagulation Factor Xa”, Science, 248:593-596 (1990).

Factor Xa inhibitory compounds which are not large polypeptide-type inhibitors have also been reported including: Tidwell, R. R. et al., “Strategies for Anticoagulation With Synthetic Protease Inhibitors. Xa Inhibitors Versus Thrombin Inhibitors”, Thromb. Res., 19:339-349 (1980); Turner, A. D. et al., “p-Amidino Esters as Irreversible Inhibitors of Factor IXa and Xa and Thrombin”, Biochemistry, 25:4929-4935 (1986); Hitomi, Y. et al., “Inhibitory Effect of New Synthetic Protease Inhibitor (FUT-175) on the Coagulation System”, Haemostasis, 15:164-168 (1985); Sturzebecher, J. et al., “Synthetic Inhibitors of Bovine Factor Xa and Thrombin. Comparison of Their Anticoagulant Efficiency”, Thromb. Res., 54:245-252 (1989); Kam, C. M. et al., “Mechanism Based Isocoumarin Inhibitors for Trypsin and Blood Coagulation Serine Proteases: New Anticoagulants”, Biochemistry, 27:2547-2557 (1988); Hauptmann, J. et al., “Comparison of the Anticoagulant and Antithrombotic Effects of Synthetic Thrombin and Factor Xa Inhibitors”, Thromb. Haemost., 63:220-223 (1990); and the like.

Others have reported Factor Xa inhibitors which are small molecule organic compounds, such as nitrogen containing heterocyclic compounds which have amidino substituent groups, wherein two functional groups of the compounds can bind to Factor Xa at two of its active sites. For example, WO 98/28269 describes pyrazole compounds having a terminal C(═NH)—NH₂ group; WO 97/21437 describes benzimidazole compounds substituted by a basic radical which are connected to a naphthyl group via a straight or branched chain alkylene, —C(═O) or —S(═O)₂ bridging group; WO 99/10316 describes compounds having a 4-phenyl-N-alkylamidino-piperidine and 4-phenoxy-N-alkylamidino-piperidine group connected to a 3-amidinophenyl group via a carboxamidealkyleneamino bridge; and EP 798295 describes compounds having a 4-phenoxy-N-alkylamidino-piperidine group connected to an amidinonaphthyl group via a substituted or unsubstituted sulfonamide or carboxamide bridging group.

There exists a need for effective therapeutic agents for the regulation of hemostasis, and for the prevention and treatment of thrombus formation and other pathological processes in the vasculature induced by thrombin such as restenosis and inflammation. In particular, there continues to be a need for compounds which selectively inhibit factor Xa or its precursors. Compounds that have different combinations of bridging groups and functional groups than compounds previously discovered are needed, particularly compounds which selectively or preferentially bind to Factor Xa. Compounds with a higher degree of binding to Factor Xa than to thrombin are desired, especially those compounds having good bioavailability and/or solubility.

### SUMMARY OF THE INVENTION

The present invention relates to novel compounds which inhibit factor Xa, their pharmaceutically acceptable isomers, salts, hydrates, solvates and prodrug derivatives, and pharmaceutically acceptable compositions thereof which have particular biological properties and are useful as potent and specific inhibitors of blood coagulation in mammals. In another aspect, the invention relates to methods of using these inhibitors as diagnostic reagents or as therapeutic agents for disease states in mammals which have coagulation disorders, such as in the treatment or prevention of any thrombotically mediated acute coronary or cerebrovascular syndrome, any thrombotic syndrome occurring in the venous system, any coagulopathy, and any thrombotic complications associated with extracorporeal circulation or instrumentation, and for the inhibition of coagulation in biological samples.

In certain embodiments, this invention relates to novel compounds which are potent and highly selective inhibitors of isolated factor Xa when assembled in the prothrombinase complex. These compounds show selectivity for factor Xa versus other proteases of the coagulation cascade (e.g. thrombin, etc.) or the fibrinolytic cascade, and are useful as diagnostic reagents as well as antithrombotic agents.

In a preferred embodiment, the present invention provides a compound of the formula I:

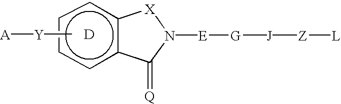

wherein:

A is selected from:

(a) C₁-C₆-alkyl;

(b) C₃-C₈-cycloalkyl;

(c) —N(R,R¹), (R,R¹)N—C(═NR²)—, R¹—C(═NR²)—, (R,R¹)N—C(═NR²)—NR³—, R—C(═NR²)—N(R³)—.;

(d) phenyl, which is independently substituted with 0-2 R¹ substituents;

(e) naphthyl, which is independently substituted with 0-2 R¹ substituents; and

(f) a monocyclic or fused bicyclic heterocyclic ring system having from 5 to 10 ring atoms, wherein 1-4 ring atoms of the ring system are selected from N, O and S, and wherein the ring system is optionally substituted with 0-2 R¹ substituents;

R and R¹ are independently selected from H, halo, C₁₋₄alkyl, C₂₋₆alkenyl, C₂₋₆alkynyl, C₃₋₈cycloalkyl, C₀₋₄alkylC₃₋₈cycloalkyl, C₀₋₄alkylphenyl, C₀₋₄alkylnaphthyl, —CN, —NO₂, (CH₂)ₘCON(R²,R³), (CH₂)ₘCO₂R², (CH₂)ₘN(R²,R³), (CH₂)ₘSO₂N(R²,R³), (CH₂)ₘSO₂R², CF₃, OR², N(R²,R³), (R²,R³)N—C(═NR⁴)—, R²—C(═NR⁴)—, and a 3-8 membered cyclic system containing from 0-4 heteroatoms selected from N, O and S, wherein from 1-4 hydrogen atoms on the ring system are optionally independently replaced with a member selected from the group consisting of halo, C₁-C₄-alkyl, C₁₋₄alkyl, C₂₋₆alkenyl, C₂₋₆alkynyl, C₃₋₈cycloalkyl, C₀₋₄alkylC₃₋₈cycloalkyl, —CN and —NO₂; wherein R and R¹ taken together may form a ring;

m is an integer of 0-4;

R² and R³ are independently selected from H, C₁₋₄alkyl, C₂₋₆alkenyl, C₂₋₆alkynyl, C₃₋₈cycloalkyl, C₀₋₄alkylC₃₋₈cycloalkyl, OH, NH₂, OC₁₋₄alkyl, N(C₁₋₄alkyl, C₁₋₄alkyl), C₀₋₄alkylphenyl and C₀₋₄alkylnaphthyl, wherein from 1-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂; R² and R³ taken together may form a ring;

Y is selected from a direct link, —C(═O)—, —CH₂—, —N(R⁴)—CH₂—, —CH₂N(R⁴)—, —N(R⁴)—, —C(═O)—N(R⁴)—, —N(R⁴)—C(═O)—, —C(═NR⁴)—, —C(═NR⁴)—N(R)—, —C(═NR⁴)—CH₂—, —C(═NR⁴)—N(R^{4a})—CH₂—, —S(═O)₂—, —S(═O)—, —O—, —S—, —SO₂—N(R⁴)— and —N(R⁴)—SO₂—;

R⁴ and R^{4a} are independently selected from H, C₁₋₄alkyl, C₂₋₆alkenyl, C₂₋₆alkynyl, C₃₋₈cycloalkyl, C₀₋₄alkylC₃₋₈cycloalkyl, C₀₋₄alkylphenyl and C₀₋₄alkylnaphthyl, wherein from 1-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂;

D is a member selected from phenyl, which is independently substituted with 0-2 R^{1a} substituents; and an aromatic five or six-membered heterocyclic ring having from 1-2 ring hetero atoms selected from oxygen, sulfur and nitrogen atoms, and wherein the ring atoms are optionally substituted with 0-2 R^{1a} substituents;

R^{1a} is selected from H, halo, C₁₋₄alkyl, C₂₋₆alkenyl, C₂₋₆alkynyl, C₃₋₈cycloalkyl, C₀₋₄alkylC₃₋₈cycloalkyl, —CN, —NO₂, (CH₂)ₙN(R^{2a},R^{3a}), (CH₂)ₙSO₂N(R^{2a},R^{3a}), (CH₂)ₙSO₂R^{2a}, (CH₂)ₙOR^{2a}, (CH₂)ₙCON(R^{2a},R^{3a}), (CH₂)ₙCO₂R^{2a}, CF₃, and a 5-6 membered aromatic heterocyclic system containing from 1-4 heteroatoms selected from N, O and S, wherein from 0-4 hydrogen atoms on the aromatic heterocyclic system are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂;

n is an integer of 0-4;

R^{2a} and R^{3a}are independently selected from H, C₁₋₄alkyl, C₂₋₆alkenyl, C₂₋₆alkynyl, C₃₋₈cycloalkyl, C₀₋₄alkylC₃₋₈cycloalkyl, C₀₋₄alkylphenyl and C₀₋₄alkylnaphthyl, wherein from 0-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂;

X is a part of a 5-8 membered ring, and is preferably selected from —C(R⁵,R^{5a})—C(═O)—, —C(R⁵,R^{5a})_C(═S)—, —C(R⁵,R^{5a})—, —C(R⁵,R^{5a})—C(R⁶,R^{6a})_, —C(R⁵,R^{5a})_C(R⁶,R^{6a})_C(R⁷,R^{7a})—, —C(═O)—, —S(═O)₂—, —C(R⁵)═C(R⁶)—C(═O)—, —C(R⁵)═C(R⁶)—C(═S)—, —C(R⁵)═C(R⁶)—, —O—C(R⁵,R^{5a})—C(═O)—, —S(═O)—, —O—C(R⁵,R^{5a})—C(═S)—, —S—C(R⁵,R^{5a})—C(═O)—, —S—C(R⁵,R^{5a})—C(═S)—, N═C(R⁵)—C(═S)—, —N═C(R⁵)—C(═O)—, —O—C(R⁵,R^{5a})—C(R⁶,R^{6a}), —N(R⁴)—C(R⁵,R^{5a})—C(R⁶,R^{6a})—, —N(R⁴)—C(R⁵,R^{5a})—, —O—C(R⁵,R^{5a})—, —N═C(R⁵)—; —S(═O)—C(R⁵,R^{5a})—C(R⁶,R^{6a})—, —S(═O)₂—C(R⁵,R^{5a})—C(R⁶,R^{6a})—, —C(═C(R^{5a},R^{5b}))—C(═O)— and —C(═C(R^{5a},R^{5b}))—C(═S)—; wherein the first named atom of the chain is directly attached to D, and wherein D, X and the N atom attached to the last chain atom of X collectively form a bicyclic ring structure;

R⁵, R^{5a}, R⁶, R^{6a}, R⁷, and R^{7a} are independently selected from H, C₁₋₄alkyl, C₂₋₆alkenyl, C₂₋₆alkynyl, C₃₋₈cycloalkyl, C₀₋₄alkylC₃₋₈cycloalkyl, NO₂, CF₃, (CH₂)ₙOC₁₋₄alkyl, (CH₂)ₙN(C₁₋₄alkyl, C₁₋₄alkyl), (CH₂)ₙCO₂C₁₋₄alkyl, (CH₂)ₙCON(C₁₋₄alkyl, C₁₋₄alkyl), C₀₋₄alkylphenyl and C₀₋₄alkylnaphthyl, wherein from 0-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂; where two alkyl groups may form a ring and n is as defined before;

Q is a member selected from ═H₂ and ═O;

E is a member selected from a direct link, —C(R⁸,R^{8a})—, —C(R⁸,R^{8a})C(R⁹,R^{9a})—, —C(R⁸,R^{8a})C(R⁹,R^{9a})C(R¹⁰,R^{10a})— and —C(═O)—;

R⁸, R^{8a}, R⁹, R^{9a}, R¹⁰ and R^{10a} are independently a member selected from H, C₁₋₄alkyl, C₂₋₆alkenyl, C₂₋₆alkynyl, C₃₋₈cycloalkyl, C₀₋₄alkyl-C₃₋₈cycloalkyl, C₀₋₄alkylphenyl, C₀₋₄alkylnaphthyl, —C₀₋₄alkylCO₂R¹¹, —C₀₋₄alkylC(═O)N(R¹¹,R^{11a}), —C₀₋₄alkylOC₀₋₄alkylR¹¹; —CH₂—CH₂—O—R¹¹, —N(—CH₂—CH₂—O—R¹¹)₂, —C₀₋₄alkylN(R¹¹)C(═O)R¹², —C₀₋₄alkylN(R¹¹)SO₂R¹², C₀₋₄alkylOH, C₀₋₄alkylNR¹¹R^{11a}, C₀₋₄alkylOC₁₋₄alkyl, C₀₋₄alkylN(C₁₋₄alkyl, C₁₋₄alkyl) and a naturally occurring or synthetic amino acid side chain, wherein from 1-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkyl-C₃₋₈cycloalkyl, —CN and —NO₂; R⁸ and R⁹, or R⁹ and R¹⁰, or R⁸ and R^{8a}, or R⁹ and R^{9a} taken together may form a ring;

R¹¹, R^{11a} and R¹² are independently selected from H, C₁₋₄alkyl, C₀₋₄alkylCON(R¹³,R¹⁴), C₀₋₄alkylCOR¹³, C₀₋₄alkylN(R¹³,R¹⁴) and C₀₋₄alkylOR¹³; R¹¹ and R^{11a}, taken together with N, may form a 5-8 membered heterocyclic ring containing 1-4 heteroatoms selected from N, O and S;

R¹³ and R¹⁴ are independently selected from H and C₁₋₄alkyl; or R¹³ and R¹⁴ taken together with N form a 5-8 membered heterocyclic ring containing 1-4 heteroatoms selected from N, O and S;

G is a member selected from a direct link, —O—, —O—C(R¹⁵,R^{15a})—, —N(R¹⁵)—, —N(R¹⁵)—C(R^{15a},R^{15b})—, —S—, —N(R¹⁵)—S(═O)—, —N(R¹⁵)—S(═O)₂—, —S(═O)—N(R¹⁵)—, —S(═O)₂N(R¹⁵)—, —N(R¹⁵)—C(═O)—, —C(═O)—N(R¹⁵)—, —N(R¹⁵)—C(═O)—N(R^{15a})— and a monocyclic aromatic or non-aromatic heterocyclic ring having from 5 to 8 ring atoms, wherein 0-4 ring atoms of the ring system are selected from N, O and S;

R¹⁵, R^{15a} and R^{15b} are independently selected from H, C₁₋₄alkyl, C₂₋₆alkenyl, C₂₋₆alkynyl, C₃₋₈cycloalkyl, C₀₋₄alkylC₃₋₈cycloalkyl, C₀₋₄alkylphenyl, C₀₋₄alkylnaphthyl, C₀₋₄alkylheteroaryl, C₁₋₄alkylCO₂H, C₁₋₄alkylCO₂C₁₋₄alkyl, C₁₋₄alkylCONH₂, C₁₋₄alkylCON(C₁₋₄alkyl, C₁₋₄alkyl), C₂₋₄alkylOH, C₂₋₄alkylNH₂, C₂₋₄alkylOC₁₋₄alkyl and C₂₋₄alkylN(C₁₋₄alkyl, C₁₋₄alkyl), wherein from 1-4 hydrogen atoms on the ring atoms of the phenyl, naphthyl and heteroaryl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂;

J is a member selected from a direct link, —O—, —S—, —N(R¹⁶)—, —N(R¹⁶)—C(═O)—, —C(═O)—N(R¹⁶)—, —N(R¹⁶)—CH₂—, —S(═O)₂, —S(═O)— and —OCH₂—;

R¹⁶ is selected from H, C₁₋₄alkyl, C₂₋₆alkenyl, C₂₋₆alkynyl, C₃₋₈cycloalkyl, C₀₋₄alkyl-C₃₋₈cycloalkyl, C₀₋₄alkylphenyl, C₀₋₄alkylnaphthyl, C₀₋₄alkylheterocyclic ring having from 1 to 4 hetero ring atoms selected from the group consisting of N, O and S, —CH₂CO₂C₁₋₄alkyl-, CH₂CO₂H, CH₂CON(C₁₋₄alkyl, C₁₋₄alkyl), CH₂CONH₂, C(═O)C₁₋₄alkyl, SO₂C₁₋₄alkyl, CH₂CO₂—C₁₋₄alkylphenyl and CH₂CO₂C₁₋₄alkylnaphthyl;

Z is selected from phenyl, which is independently substituted with 0-2 R^{1b} substituents; naphthyl, which is independently substituted with 0-2 R^{1b} substituents; and a monocyclic or fused bicyclic heterocyclic ring system having from 5 to 10 ring atoms, wherein 1-4 ring atoms of the ring system are selected from N, O and S, and wherein the ring system is optionally substituted from 0-2 R^{1b} substituents;

R^{1b} is selected from H, halo, C₁₋₄alkyl, C₂₋₆alkenyl, C₂₋₆alkynyl, C₃₋₈cycloalkyl, C₀₋₄alkylC₃₋₈cycloalkyl, —CN, —NO₂, N(R^{2b},R^{3b}), SO₂N(R^{2b},R^{3b}), SO₂R^{2b}, CO₂N(R^{2b},R^{3b}), CO₂R^{2b}, CF₃, OR^{2b}, O—CH₂—CH₂—OR^{2b}, O—CH₂—CH₂—N(R^{2b},R^{3b}), O—CH₂—CON(R^{2b},R^{3b}), O—CH₂—CH₂—N(R^{2b},R^{3b}), O—CH₂—COOR^{2b}, N(R^{2b})—CH₂—CH₂—OR^{3b}, N(—CH₂—CH₂—OR^{2b})₂, N(R^{2b})—C(═O)R^{3b}, N(R^{2b})—SO₂—R^{3b} and a 5-6 membered aromatic heterocyclic system containing from 1-4 heteroatoms selected from N, O and S, wherein from 1-4 hydrogen atoms on the aromatic heterocyclic system are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂;

R^{2b} and R^{3b}are independently selected from H, C₁₋₄alkyl, C₂₋₆alkenyl, C₂₋₆alkynyl, C₃₋₈cycloalkyl, C₀₋₄alkylC₃₋₈cycloalkyl, C₀₋₄alkylphenyl and C₀₋₄alkylnaphthyl, wherein from 1-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂;

L is selected from H, —CN, C(═O)N(R¹⁷,R^{17a}), (CH₂)ₙN(R¹⁷,R^{17a}R), C(═NR¹⁷)N(R^{17a},R^{17b}), OR¹⁷, —NR¹⁷C(═NR^{17a})N(R^{17b},R^{17c}) and NR¹⁷C(═NR^{17a})—R^{17b};

R¹⁷, R^{17a}, R^{17b}, and R^{17c} are independently selected from H, —OR¹⁸, —NR¹⁸R^{18a}, C₁₋₄alkyl, C₀₋₄alkylphenyl, C₀₋₄alkylnaphthyl, COOC₁₋₄alkyl, COO—C₀₋₄alkylphenyl and COO—C₀₋₄alkylnaphthyl, wherein from 1-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN, and —NO₂;

R¹⁸ and R^{18a} are independently selected from H, C₁₋₄alkyl, C₂₋₆alkenyl, C₂₋₆alkynyl, C₃₋₈cycloalkyl, C₀₋₄alkylC₃₋₈cycloalkyl, C₀₋₄alkylphenyl and C₀₋₄alkylnaphthyl, wherein from 1-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN, and —NO₂;

and all pharmaceutically acceptable isomers, salts, hydrates, solvates and prodrug derivatives thereof.

In certain aspects of this invention, compounds are provided which are useful as diagnostic reagents. In another aspect, the present invention includes pharmaceutical compositions comprising a pharmaceutically effective amount of the compounds of this invention and a pharmaceutically acceptable carrier. In yet another aspect, the present invention includes methods comprising using the above compounds and pharmaceutical compositions for preventing or treating disease states characterized by undesired thrombosis or disorders of the blood coagulation process in mammals, or for preventing coagulation in biological samples such as, for example, stored blood products and samples. Optionally, the methods of this invention comprise administering the pharmaceutical composition in combination with an additional therapeutic agent such as an antithrombotic and/or a thrombolytic agent and/or an anticoagulant.

The preferred compounds also include their pharmaceutically acceptable isomers, hydrates, solvates, salts and prodrug derivatives.

### BRIEF DESCRIPTION OF THE DRAWINGS

Not applicable

### DETAILED DESCRIPTION OF THE INVENTION

Definitions

In accordance with the present invention and as used herein, the following terms are defined with the following meanings, unless explicitly stated otherwise.

The term “alkenyl” refers to a trivalent straight chain or branched chain unsaturated aliphatic radical. The term “alkynyl” refers to a straight or branched chain aliphatic radical that includes at least two carbons joined by a triple bond. If no number of carbons is specified, alkenyl and alkynyl each refer to radicals having from 2-12 carbon atoms.

The term “alkyl” refers to saturated aliphatic groups including straight-chain, branched-chain and cyclic groups having the number of carbon atoms specified, or if no number is specified, having up to 12 carbon atoms. The term “cycloalkyl” as used herein refers to a mono-, bi-, or tricyclic aliphatic ring having 3 to 14 carbon atoms and preferably 3 to 7 carbon atoms. In a broader term, alkyl may be hydrogen and may also have heteroatom containing substitution groups.

As used herein, the terms “carbocyclic ring structure” and “C₃₋₁₆ carbocyclic mono, bicyclic or tricyclic ring structure” or the like are each intended to mean stable ring structures having only carbon atoms as ring atoms wherein the ring structure is a substituted or unsubstituted member selected from the group consisting of: a stable monocyclic ring which is aromatic ring (“aryl”) having six ring atoms; a stable monocyclic non-aromatic ring having from 3 to 7 ring atoms in the ring; a stable bicyclic ring structure having a total of from 7 to 12 ring atoms in the two rings wherein the bicyclic ring structure is selected from the group consisting of ring structures in which both of the rings are aromatic, ring structures in which one of the rings is aromatic and ring structures in which both of the rings are non-aromatic; and a stable tricyclic ring structure having a total of from 10 to 16 atoms in the three rings wherein the tricyclic ring structure is selected from the group consisting of: ring structures in which three of the rings are aromatic, ring structures in which two of the rings are aromatic and ring structures in which three of the rings are non-aromatic. In each case, the non-aromatic rings when present in the monocyclic, bicyclic or tricyclic ring structure may independently be saturated, partially saturated or fully saturated. Examples of such carbocyclic ring structures include, but are not limited to, cyclopropyl, cyclobutyl, cyclopentyl, cyclohexyl, adamantyl, cyclooctyl, [3.3.0]bicyclooctane, [4.3.0]bicyclononane, [4.4.0]bicyclodecane (decalin), 2.2.2]bicyclooctane, fluorenyl, phenyl, naphthyl, indanyl, adamantyl, or tetrahydronaphthyl (tetralin). Moreover, the ring structures described herein are optionally attached to one or more indicated pendant groups via any carbon atom which results in a stable structure. The term “substituted” as used in conjunction with carbocyclic ring structures means that hydrogen atoms attached to the ring carbon atoms of ring structures described herein are optionally substituted by one or more of the substituents indicated for that structure if such substitution(s) would result in a stable compound.

The term “aryl” which is included with the term “carbocyclic ring structure” refers to an unsubstituted or substituted aromatic ring, substituted with one, two or three substituents selected from loweralkoxy, loweralkyl, loweralkylamino, hydroxy, halogen, cyano, hydroxyl, mercapto, nitro, thioalkoxy, carboxaldehyde, carboxyl, carboalkoxy and carboxamide, including but not limited to carbocyclic aryl, heterocyclic aryl, and biaryl groups and the like, all of which are optionally substituted. Preferred aryl groups include phenyl, halophenyl, loweralkylphenyl, naphthyl, biphenyl, phenanthrenyl and naphthacenyl.

The term “arylalkyl” which is included with the term “carbocyclic aryl” refers to one, two, or three aryl groups having the number of carbon atoms designated, appended to an alkyl group having the number of carbon atoms designated. Suitable arylalkyl groups include, but are not limited to, benzyl (Bn), picolyl, naphthylmethyl, phenethyl, benzyhydryl, trityl, and the like, all of which are optionally substituted.

As used herein, the term “heterocyclic ring” or “heterocyclic ring system” is intended to mean a substituted or unsubstituted member selected from the group consisting of stable monocyclic ring having from 5-7 members in the ring itself and having from 1 to 4 hetero ring atoms selected from the group consisting of N, O and S; a stable bicyclic ring structure having a total of from 7 to 12 atoms in the two rings wherein at least one of the two rings has from 1 to 4 hetero atoms selected from N, O and S, including bicyclic ring structures wherein any of the described stable monocyclic heterocyclic rings is fused to a hexane or benzene ring; and a stable tricyclic heterocyclic ring structure having a total of from 10 to 16 atoms in the three rings wherein at least one of the three rings has from 1 to 4 hetero atoms selected from the group consisting of N, O and S. Any nitrogen and sulfur atoms present in a heterocyclic ring of such a heterocyclic ring structure may be oxidized. Unless indicated otherwise the terms “heterocyclic ring” or “heterocyclic ring system” include aromatic rings, as well as non-aromatic rings which can be saturated, partially saturated or fully saturated non-aromatic rings. Also, unless indicated otherwise the term “heterocyclic ring system” includes ring structures wherein all of the rings contain at least one hetero atom as well as structures having less than all of the rings in the ring structure containing at least one hetero atom, for example bicyclic ring structures wherein one ring is a benzene ring and one of the rings has one or more hetero atoms are included within the term “heterocyclic ring systems” as well as bicyclic ring structures wherein each of the two rings has at least one hetero atom. Moreover, the ring structures described herein may be attached to one or more indicated pendant groups via any hetero atom or carbon atom which results in a stable structure. Further, the term “substituted” means that one or more of the hydrogen atoms on the ring carbon atom(s) or nitrogen atom(s) of the each of the rings in the ring structures described herein are optionally replaced by one or more of the indicated substituents if such replacement(s) would result in a stable compound. Nitrogen atoms in a ring structure are optionally quaternized, but such compounds are specifically indicated or are included within the term “a pharmaceutically acceptable salt” for a particular compound. When the total number of O and S atoms in a single heterocyclic ring is greater than 1, it is preferred that such atoms not be adjacent to one another. Preferably, there are no more that 1 O or S ring atoms in the same ring of a given heterocyclic ring structure.

Examples of monocylic and bicyclic heterocylic ring systems, in alphabetical order, are acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolinyl, indolizinyl, indolyl, 3H-indolyl, isobenzofuranyl, isochromanyl, isoindazolyl, isoindolinyl, isoindolyl, isoquinolinyl (benzimidazolyl), isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyroazolidinyl, pyrazolinyl, pyrazolyl, pyridazinyl, pryidooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadazinyl, 1,2,3-thiadiazolyl, 1,2,4-thiadiazolyl, 1,2,5-thiadiazolyl, 1,3,4-thiadiazolyl, thianthrenyl, tlhiazolyl, thienyl, thienothiazolyl, thienooxazolyl, thienoimidazolyl, thiophenyl, trialzinyl, 1,2,3-triazolyl, 1,2,4-triazolyl, 1,2,5-triazolyl, 1,3,4-triazolyl and xanthenyl. Preferred heterocyclic ring structures include, but are not limited to, pyridinyl, furanyl, thienyl, pyrrolyl, pyrazolyl, pyrrolidinyl, imidazolyl, indolyl, benzimidazolyl, 1H-indazolyl, oxazolinyl, or isatinoyl. Also included are fused ring and spiro compounds containing, for example, the above heterocylic ring structures.

As used herein the term “aromatic heterocyclic ring system” has essentially the same definition as for the monocyclic and bicyclic ring systems except that at least one ring of the ring system is an aromatic heterocyclic ring or the bicyclic ring has an aromatic or non-aromatic heterocyclic ring fused to an aromatic carbocyclic ring structure.

The terms “halo” or “halogen” as used herein refer to Cl, Br, F or I substituents. The term “haloalkyl”, and the like, refer to an aliphatic carbon radicals having at least one hydrogen atom replaced by a Cl, Br, F or I atom, including mixtures of different halo atoms. Trihaloalkyl includes trifluoromethyl and the like as preferred radicals, for example.

The term “methylene” refers to —CH₂—.

The term “a naturally occurring or synthetic amino acid side chain” refers to the group R in the general formula of an alpha amino acid R—C(H)(NH₂)—CO₂H. A naturally occurring amino acid is one of those that are commonly found as building blocks in natural proteins. A synthetic amino acid is one of those that contain either an R group or an absolute stereochemistry that are not commonly found among the naturally occurring amino acids.

The term “pharmaceutically acceptable salts” includes salts of compounds derived from the combination of a compound and an organic or inorganic acid. These compounds are useful in both free base and salt form. In practice, the use of the salt form amounts to use of the base form; both acid and base addition salts are within the scope of the present invention.

“Pharmaceutically acceptable acid addition salt” refers to salts retaining the biological effectiveness and properties of the free bases and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicyclic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium and magnesium salts. Salts derived from pharmaceutically acceptable organic nontoxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particularly preferred organic nontoxic bases are isopropylamine, diethylamine, ethanolamine, trimethamine, dicyclohexylamine, choline, and caffeine.

“Biological property” for the purposes herein means an in vivo effector or antigenic function or activity that is directly or indirectly performed by a compound of this invention that are often shown by in vitro assays. Effector functions include receptor or ligand binding, any enzyme activity or enzyme modulatory activity, any carrier binding activity, any hormonal activity, any activity in promoting or inhibiting adhesion of cells to an extracellular matrix or cell surface molecules, or any structural role. Antigenic functions include possession of an epitope or antigenic site that is capable of reacting with antibodies raised against it.

In the compounds of this invention, carbon atoms bonded to four non-identical substituents are asymmetric. Accordingly, the compounds may exist as diastereoisomers, enantiomers or mixtures thereof. The syntheses described herein may employ racemates, enantiomers or diastereomers as starting materials or intermediates. Diastereomeric products resulting from such syntheses can be separated by chromatographic or crystallization methods, or by other methods known in the art. Likewise, enantiomeric product mixtures can be separated using the same techniques or by other methods known in the art. Each of the asymmetric carbon atoms, when present in the compounds of this invention, are in one of two configurations (R or S) and both are within the scope of the present invention.

Preferred Embodiments

In a preferred embodiment, the present invention provides a compound according to the formula I:

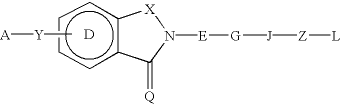

wherein:

A is selected from:

(a) C₁-C₆-alkyl;

(b) C₃-C₈-cycloalkyl;

(c) —N(RR¹), (R,R¹)N—C(═NR²)—, R¹—C(═NR²)—, (R,R¹)N—C(═NR²)—N(R³)—, R—C(═NR²)—N(R³)_;

(d) phenyl, which is independently substituted with 0-2 R¹ substituents;

(e) naphthyl, which is independently substituted with 0-2 R¹ substituents; and

(f) a monocyclic or fused bicyclic heterocyclic ring system having from 5 to 10 ring atoms, wherein 1-4 ring atoms of the ring system are selected from N, O and S, and wherein the ring system is optionally substituted with 0-2 R¹ substituents;

R and R¹ are independently selected from H, halo, C₁₋₄alkyl, —CN, —NO₂, (CH₂)ₘCON(R²,R³), (CH₂)ₘCO₂R², (CH₂)ₘN(R²,R³), SO₂N(R²,R³), SO₂R², CF₃, OR², N(R²,R³), (R²,R³)N—C(═NR⁴)—, R²—C(═NR⁴)— and a 3-8 membered cyclic system containing from 0-4 heteroatoms selected from N, O and S;

m is an integer of 0-4;

R² and R³ are independently selected from H, C₁₋₄alkyl, C₂₋₆alkenyl, C₂₋₆alkynyl, C₃₋₈cycloalkyl, C₀₋₄alkylC₃₋₈cycloalkyl, OH, NH₂, OC₁₋₄alkyl, N(C₁₋₄alkyl,C₁₋₄alkyl), C₀₋₄alkylphenyl and C₀₋₄alkylnaphthyl;

Y is a member selected from a direct link, —C(═O)—, —CH₂—, —N(R⁴)—CH₂—, —CH₂N(R⁴)—, —N(R⁴)—, —C(═NR⁴)—, —C(═NR⁴)—N(R)—, —C(═NR⁴)—CH₂—, —C(═NR⁴)—N(R^{4a})—CH₂—, —O— and —S—;

R⁴ and R^{4a} are independently selected from H, C₁₋₄alkyl, C₂₋₆alkenyl, C₂₋₆alkynyl, C₃₋₈cycloalkyl, C₀₋₄alkylC₃₋₈cycloalkyl, C₀₋₄alkylphenyl and C₀₋₄alkylnaphthyl;

D is a member selected from phenyl, which is independently substituted with 0-2 R^{1a} substituents; and an aromatic five or six-membered heterocyclic ring having from 1-2 ring hetero atoms selected from oxygen, sulfur and nitrogen atoms, and wherein the ring atoms are optionally substituted with 0-2 R^{1a} substituents;

R^{1a} is selected from H, halo, C₁₋₄alkyl, —CN, —NO₂, (CH₂)ₙNR^{2a}R^{3a}, (CH₂)ₙSO₂NR^{2a}R^{3a}, (CH₂)ₙSO₂R^{2a}, (CH₂)ₙOR^{2a}, (CH₂)ₙCONR^{2a}R^{3a}, (CH₂)ₙCO₂R^{3a} and CF₃,

n is an integer of 0-4;

R^{2a} and R^{3a} are independently selected from H, C₁₋₄alkyl, C₀₋₄alkylphenyl and C₀₋₄alkylnaphthyl;

X is a part of a 5-8 membered ring, and is preferably selected from —C(R⁵,R^{5a})—C(═O), —C(R⁵,R^{5a})—, —C(R⁵,R^{5a})—C(R⁶,R^{6a})—, —C(R⁵,R^{5a})—C(R⁶,R^{6a})—C(R⁷,R^{7a})—, —C(═O)—, —S(═O)₂—, —C(R⁵)═C(R⁶)—, —S(═O)—, —O—C(R⁵,R^{5a})—C(R⁶,R^{6a})—, —N(R⁴)—C(R⁵,R^{5a})—C(R⁶,R^{6a})—, —N(R⁴)—C(R⁵,R^{5a})—, —O—C(R⁵,R^{5a})— and —N═C(R⁵)—;

R⁵, R^{5a}, R⁶, R^{6a}, R⁷, and R^{7a} are independently selected from H, C₁₋₄alkyl, NO₂, CF₃, (CH₂)ₙOC₁₋₄alkyl, (CH₂)ₙN(C₁₋₄alkyl, C₁₋₄alkyl), (CH₂)ₙCO₂C₁₋₄alkyl, (CH₂)ₙCON(C₁₋₄alkyl, C₁₋₄alkyl), C₀₋₄alkylphenyl and C₀₋₄alkylnaphthyl; two alkyl taken together may form a ring, and n is as defined before;

Q is a member selected from ═H₂ and ═O;

E is a member selected from a direct link, —C(R⁸,R^{8a})—, —C(R⁸,R^{8a})C(R⁹,R^{9a})—, —C(R⁸,R^{8a})C(R⁹,R^{9a})C(R¹⁰,R^{10a})— and —C(═O)—; wherein R⁸, R^{8a}, R⁹, R^{9a}, R¹⁰ and R^{10a} are independently selected from H, C₁₋₄alkyl, C₀₋₄alkylphenyl, C₀₋₄alkylnaphthyl, —C₀₋₄alkylCO₂R¹¹, —C₀₋₄alkylC(═O)N(R¹¹,R^{11a}), —C₀₋₄alkylOC₀₋₄alkylR¹¹, —CH₂—CH₂—O—R¹¹, —N(—CH₂—CH₂—O—R¹¹)₂, —C₀₋₄alkylN(R¹¹)C(═O)R¹², —C₀₋₄alkylN(R¹¹)SO₂R¹², C₀₋₄alkylOH, C₀₋₄alkylNH₂, C₀₋₄alkylOC₁₋₄alkyl, C₀₋₄alkylN(C₁₋₄alkyl, C₁₋₄alkyl) and a naturally occurring or synthetic amino acid side chain; wherein R¹¹, R^{11a} and R¹² are independently selected from H, C₁₋₄alkyl, C₀₋₄alkylCON(R¹³,R¹⁴), C₀₋₄alkylCOR¹³, C₀₋₄alkylN(R¹³,R¹⁴) and C₀₋₄alkylOR¹³; or R¹¹ and R^{11a} taken together with N may form a 5-8 membered ring containing 1-4 heteroatoms selected from N, O and S;

R¹³ and R¹⁴ are independently selected from H and C₁₋₄alkyl; or R¹³ and R¹⁴ taken together with N may form a 5-8 membered heterocyclic ring containing 1-4 heteroatoms selected from N, O and S;

G is selected from a direct link, —O—, —O—C(R¹⁵,R^{15a})_, —N(R⁵)—, —N(R¹⁵)—C(R^{15a},R^{15b})—, —S—, —N(R¹⁵)—S(═O)—, —N(R¹⁵)—S(═O)₂—, —S(═O)—N(R¹⁵)—, —S(═O)₂N(R¹⁵)—, —N(R¹⁵)—C(═O)—, —C(═O)—N(R¹⁵)—, —N(R¹⁵)—C(═O)—N(R^{15a})— and a monocyclic aromatic or non-aromatic ring having from 5 to 8 ring atoms, wherein 0-4 ring atoms of the ring system are selected from N, O and S;

R¹⁵, R^{15a} and R^{15b} are independently selected from H, C₁₋₄alkyl, C₀₋₄alkylphenyl, C₀₋₄alkylnaphthyl, C₁₋₄alkylCO₂H, C₁₋₄alkylCO₂C₁₋₄alkyl, C₁₋₄alkylCONH₂, C₁₋₄alkylCON(C₁₋₄alkyl, C₁₋₄alkyl), C₂₋₄alkylOH, C₂₋₄alkylNH₂, C₂₋₄alkylOC₁₋₄alkyl and C₂₋₄alkylN(C₁₋₄alkyl, C₁₋₄alkyl);

J is selected from a direct link, —O—, —S—, —N(R¹⁶)—, —N(R¹⁶)—C(═O)—, —C(═O)—N(R¹⁶)—, —N(R¹⁶)—CH₂—, —S(═O)₂—, —S(═O)— and —OCH₂—;

R¹⁶ is selected from H, C₁₋₄alkyl, C₀₋₄alkylphenyl, C₀₋₄alkylnaphthyl, CH₂CO₂C₁₋₄alkyl, CH₂CO₂H, CH₂CON(C₁₋₄alkyl, C₁₋₄alkyl), CH₂CONH₂, C(═O)C₁₋₄alkyl, SO₂C₁₋₄alkyl, CH₂CO₂—C₁₋₄alkylphenyl and CH₂CO₂C₁₋₄alkylnaphthyl;

Z is selected from phenyl, which is independently substituted with 0-2 R^{1b} substituents, naphthyl, which is independently substituted with 0-2 R^{1b} substituents and a monocyclic or fused bicyclic heterocyclic ring system having from 5 to 10 ring atoms, wherein 1-4 ring atoms of the ring system are selected from N, O and S, and wherein the ring system is optionally substituted from 0-2 R^{1b} substituents;

R^{1b} is selected from H, halo, C₁₋₄alkyl, —CN, —NO₂, N(R^{2b}R^{3b}), SO₂N(R^{2b},R^{3b}), SO₂R^{2b}, CO₂N(R^{2b},R^{3b}), CO₂R^{2b}, CF₃, OR^{2b}, O—CH₂—CH₂—OR^{2b}, O—CH₂—CH₂—N(R^{2b},R^{3b}), O—CH₂—CON(R^{2b},R^{3b}), O—CH₂—CO₂R^{2b}, N(R^{2b})—CH₂—CH₂—OR^{3b}, N(—CH₂—CH₂—OR^{2b})₂, N(R^{2b})—C(═O)R^{3b} and N(R^{2b})—SO₂—R^{3b};

R^{2b} and R^{3b} are independently selected from H, C₁₋₄alkyl, C₁₋₄alkylphenyl and C₁₋₄alkylnaphthyl;

L is selected from H, —CN, C(═O)N(R¹⁷,R^{17a}), (CH₂)ₙN(R¹⁷,R^{17a}), C(═NR¹⁷)N(R^{17a},R^{17b}), OR¹⁷, —NR¹⁷C(═NR^{17a})N(R^{17b},R^{17c}) and NR¹⁷C(═NR^{17a})—R^{17b};

R¹⁷, R^{17a}, R^{17b}, and R^{17c} are independently selected from H, —OR¹⁸, —N(R¹⁸,R^{18),} C₁₋₄alkyl, C₀₋₄alkylphenyyl and C₀₋₄alkylnaphthyl;

R¹⁸ and R^{18a} are independently selected from H, C₁₋₄alkyl, C₀₋₄alkylphenyyl and C₀₋₄alkylnaphthyl;

and all pharmaceutically acceptable isomers, salts, hydrates, solvates and prodrug derivatives thereof.

In a further preferred embodiment, the present invention provides a compound according to the formula I:

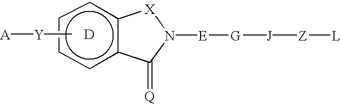

wherein:

A is a member selected from the group consisting of:

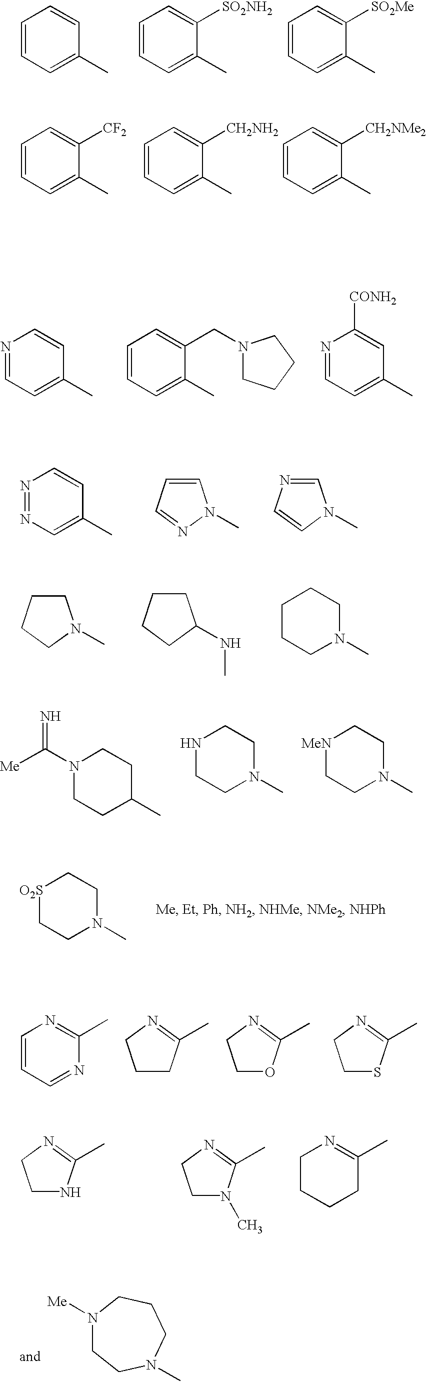

Y is selected from a direct link, —C(═O)—, —CH₂—, —NH—CH₂—, —NMe-CH₂—, —NH—, —NMe-, —C(═NH)—, —C(═NMe)-, —O— and —S—;

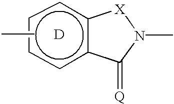

is a membered selected from the group of:

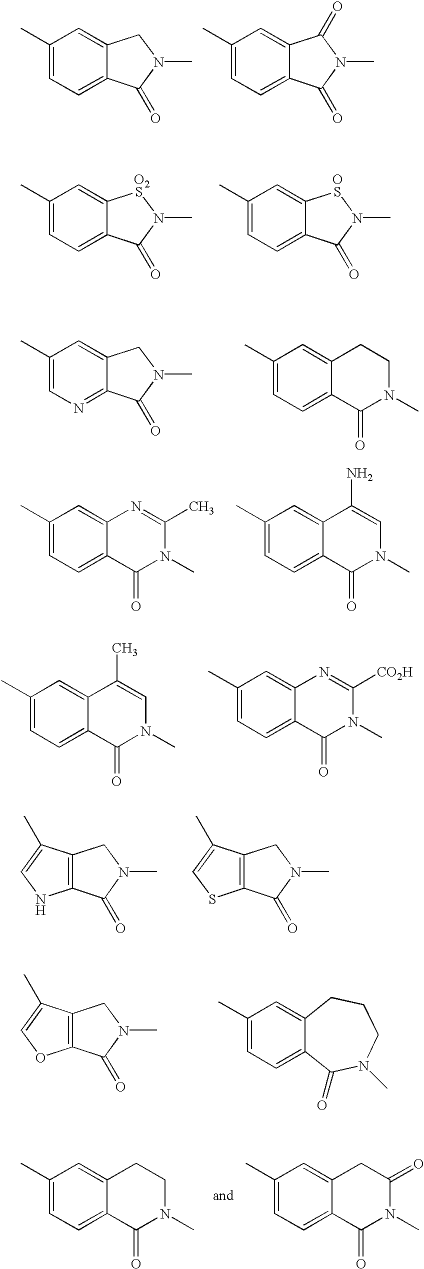

E is selected from a direct link, —CH(R⁸)—, —CH(R⁸)CH₂—, —CH(R⁸)CH₂CH₂— and —C(═O)—;

R⁸ is selected from H, OH, NH₂, NHAc, Me Et, Ph, Bn, cyclohexyl, CO₂H, CO₂Me, CONH₂, CONMe₂, CONHMe, CH₂CO₂H, CH₂CO₂Me, CH₂CONH₂, CH₂CONMe₂, CH₂CONHMe,

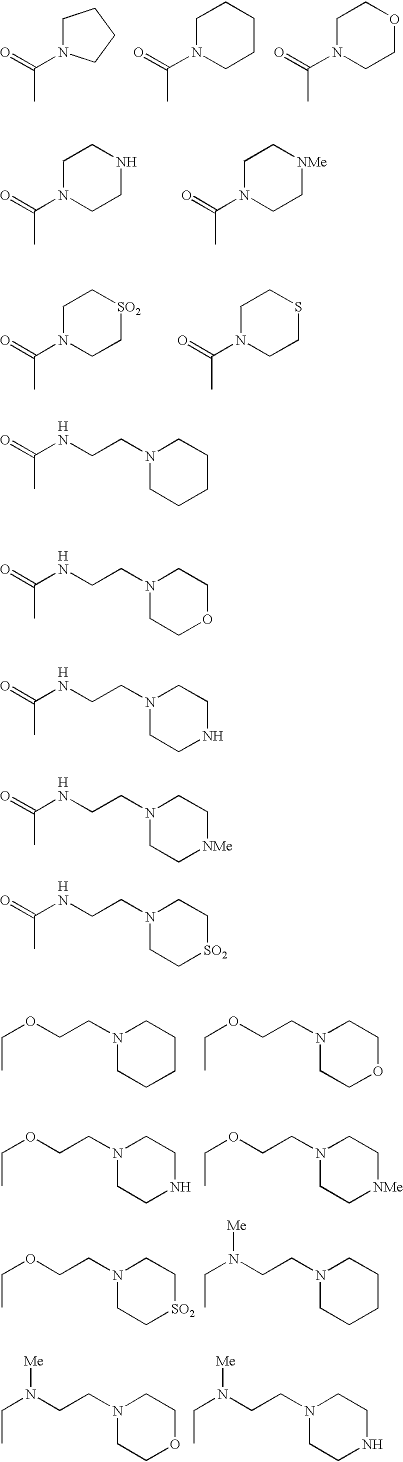

G is selected from a direct link, —O—, —N(R¹⁵)—, —S—, —N(R¹⁵)—S(═O)—, —N(R¹⁵)—S(═O)₂—, —N(R¹⁵)—C(═O)—, —C(═O)—N(R¹⁵)—, —N(R¹⁵)—C(═O)—N(R^{15a})—,

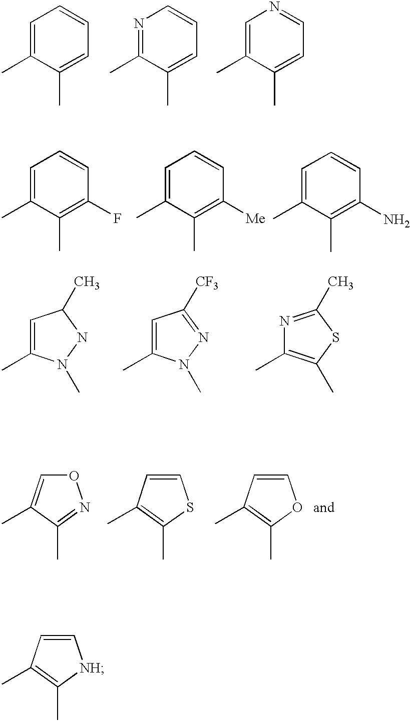

R¹⁵ and R^{15a} are independently selected from H. Me, Et, Bn, CH₂CO₂H, CH₂CO₂Me, CH₂CONH₂, CH₂CONMe₂;

J is selected from a direct link, —O—, —S—, —NH—, —NMe-, —NH—C(═O)—, —C(═O)—NH—; —NMe-C(═O)— and —C(═O)—NMe-;

Z-L together represent a member selected from

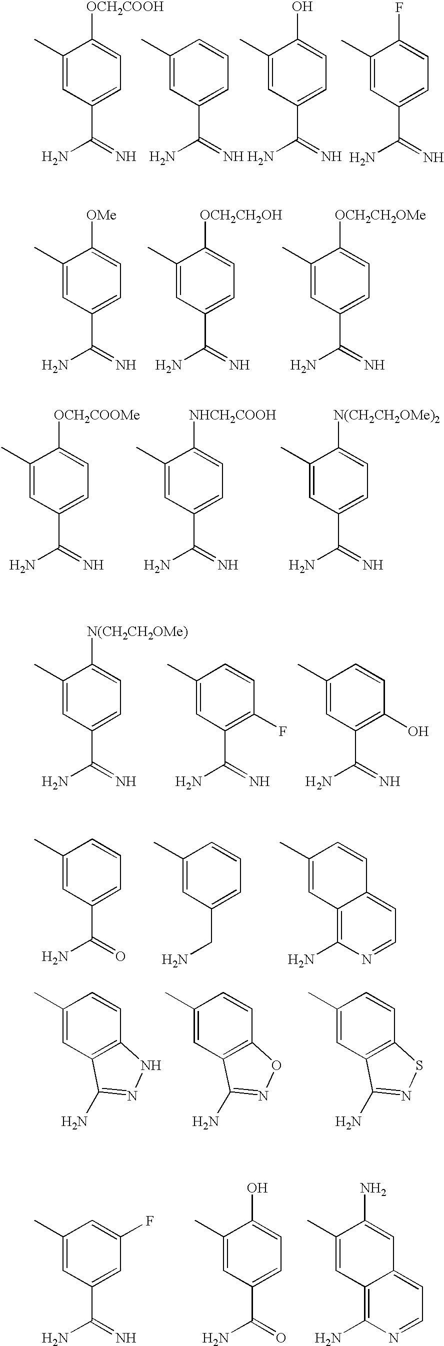

and all pharmaceutically acceptable isomers, salts, hydrates, solvates and prodrug derivatives thereof.

The following non-limiting tables illustrate representative compounds of the present invention:

TABLE 1

| | |
|---|---|
| 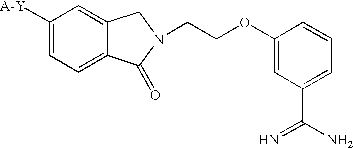 | 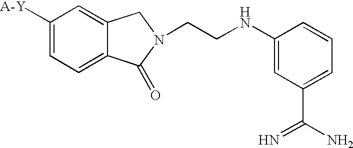 |
| 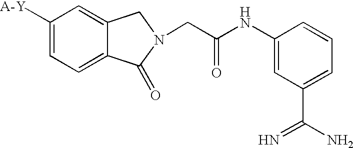 | 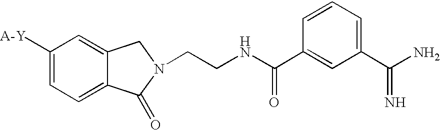 |

For the compounds of Table 1, A is selected from

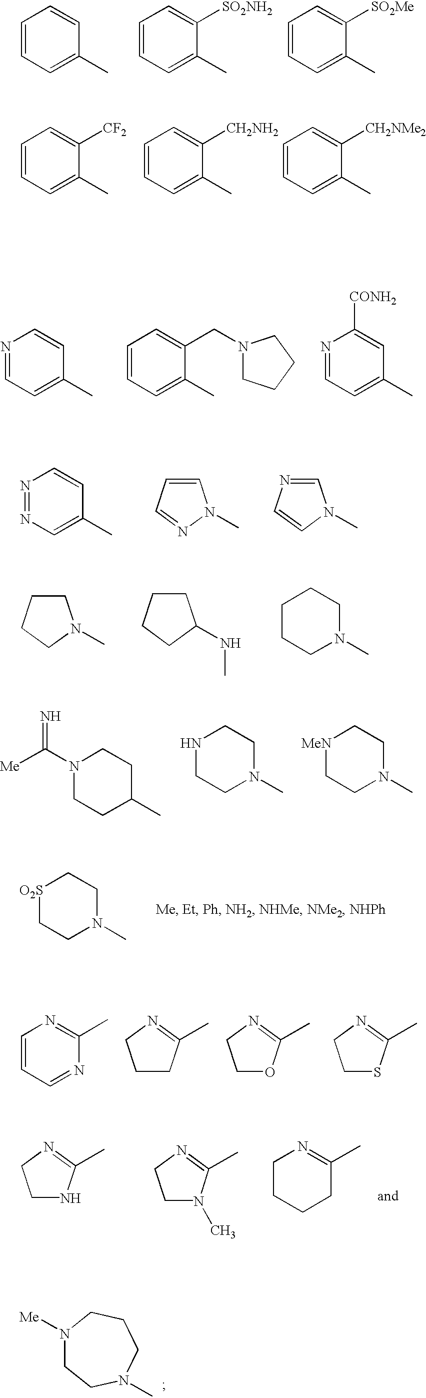

and Y is selected from a direct link, —O—, —S—, —SO₂—, —SO—, —C(═O)—, —CH₂—, —NH—CH₂—, —NMe-CH₂—, —CH₂N(R⁴)—, —C(═NH)—, —C(═NH)—CH₂—, —C(═NMe)-CH₂—, —C(═NMe)-, —NH—, —NMe-, —C(═O)—NH—, —NH—C(═O)—, —C(═NH)—NH—, —C(═NH)—NMe- and —C(═NMe)-NH—.

TABLE 2

| | |
|---|---|
| 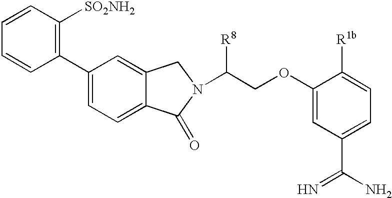 | 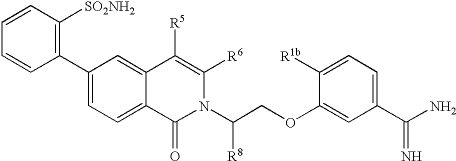 |
| 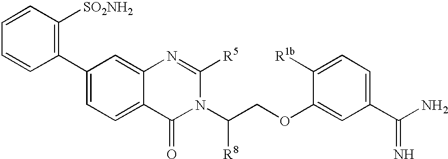 | 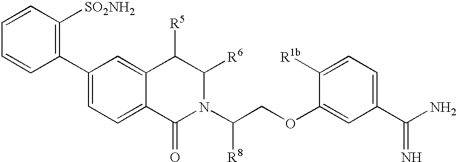 |

For the compound., of Table 2:

R^{1b} is selected from H, F, Cl, Br I, Me, OH, OMe, OPh, OBn, NH₂, OCH₂CH₂NH₂, OCH₂CH₂OH, CONH₂, NO₂, SO₂Me, SO₂NH₂, CN, CH₂OH, CH₂NH₂, CO₂H, CO₂Me, CH₂CO₂H, CH₂CO₂Me, CONMe₂ and CH₂CONMe₂;

R⁵ and R⁶ are independently selected from H, Me, Et, CF₃, Ph, Bn, CO₂H, CO₂Me, CONH₂, CONMe₂, NH₂, OH, CH₂NH₂, CH₂OH, CH₂CO₂H, CH₂CO₂Me, CH₂CONH₂ and CH₂CONMe₂; and

R⁸ is selected from H, Me, Et, Ph, Bn, cyclohexyl, CH₂cyclohexyl, CH₂NH₂, CH₂OH, CONMe₂, CONH₂, CH₂CONMe₂, CH₂CONH₂, CO₂H, CO₂Me, CH₂CO₂H, CH₂CO₂Me, naphthyl, CH₂naphthyl, CONMe₂ and CH₂CONMe₂.

TABLE 3

| | |
|---|---|
| 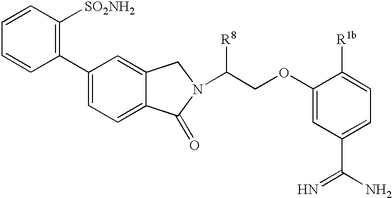 | 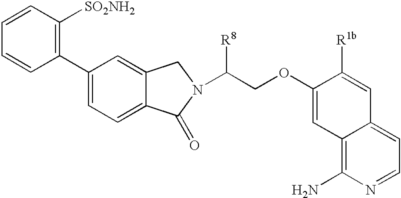 |
| 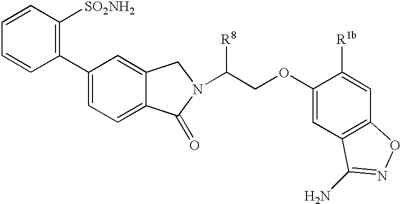 | 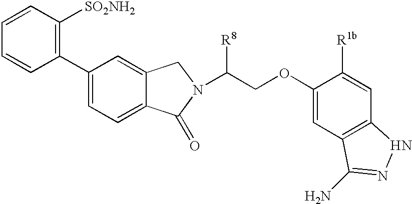 |

For the compounds of Table 3, R^{1b} is selected from H, F, Cl, Br I, Me, OH, OMe, OPh, OBn, NH₂, OCH₂CH₂NH₂, OCH₂CH₂OH, CONH₂, NO₂, SO₂Me, SO₂NH₂, CN, CH₂OH, CH₂NH₂, CO₂H, CO₂Me, CH₂CO₂H, CH₂CO₂Me, CONMe₂ and CH₂CONMe₂; and R⁸ is selected from H, Me, Et, Ph, Bn, cyclohexyl, CH₂cyclohexyl, CH₂NH₂, CH₂OH, CONMe₂, CONH₂, CH₂CONMe₂, CH₂CONH₂, CO₂H, CO₂Me, CH₂CO₂H, CH₂CO₂Me, aryl, CH₂aryl, CONMe₂ and CH₂CONMe₂.

TABLE 4

| | | |
|---|---|---|
| 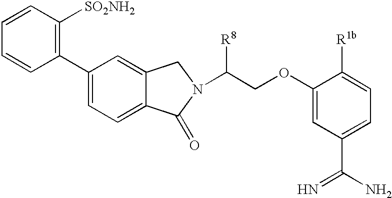 | 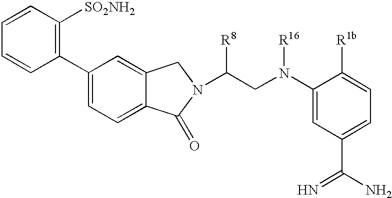 | 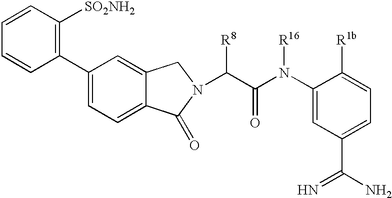 |

| | |
|---|---|
| 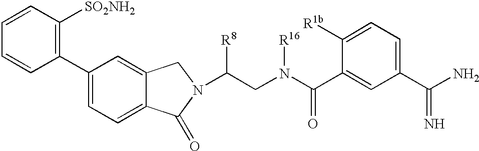 | 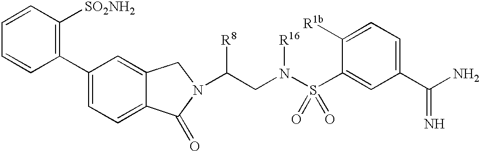 |

For the compounds of Table 4, R^{1b} is selected from H, F, Cl, Br, I, Me, OH, OMe, OPh, OBn, NH₂, OCH₂CH₂NH₂, OCH₂CH₂OH, CONH₂, NO₂, SO₂Me, SO₂NH₂, CN, CH₂OH, CH₂NH₂, CO₂H, CO₂Me, CH₂CO₂H, CH₂CO₂Me, CONMe₂ and CH₂CONMe₂; R⁸ is selected from H, Me, Et, Ph, Bn, cyclohexyl, CH₂cyclohexyl, CH₂NH₂, CH₂OH, CONMe₂, CONH₂, CH₂CONMe₂, CH₂CONH₂, CO₂H, CO₂Me, CH₂CO₂H, CH₂CO₂Me, CONMe₂ and CH₂CONMe₂; and R¹⁶ is selected from H, Me, Et, Ph, and Bn.

TABLE 5

| | |
|---|---|
| 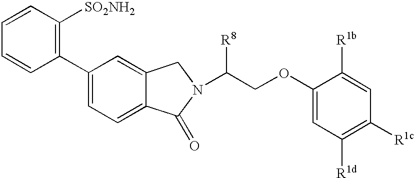 | 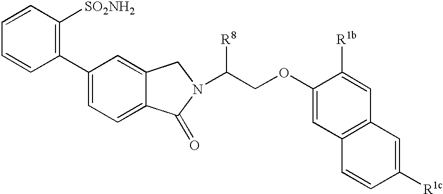 |
| 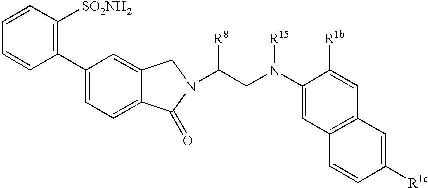 | 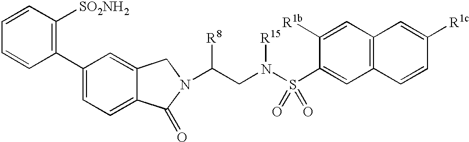 |
| 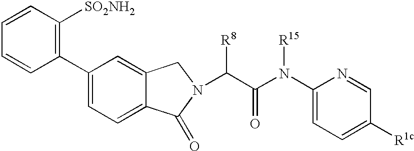 | 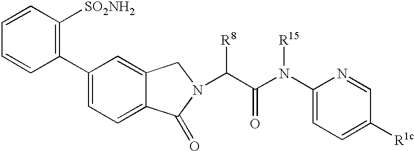 |

For the compounds of Table 5, R^{1b}, R^{1c} and R^{1d} are individually selected from H, F, Cl, Br, I, Me, OH, OMe, OPh, OBn, NH₂, OCH₂CH₂NH₂, OCH₂CH₂OH, CONH₂, NO₂, SO₂Me, SO₂NH₂, CN, CH₂OH, CH₂NH₂, CO₂H, CO₂Me, CH₂CO₂H, CH₂CO₂Me, CONMe₂ and CH₂CONMe₂; R⁸ is selected from H, Me, Et, Ph, Bn, cyclohexyl, CH₂cyclohexyl, CH₂NH₂, CH₂OH, CONMe₂, CONH₂, CH₂CONMe₂, CH₂CONH₂, CO₂H, CO₂Me, CH₂CO₂H, CH₂CO₂Me, naphthyl, CH₂naphthyl, CONMe₂ and CH₂CONMe₂; and R¹⁵ is selected from H, Me, Et, Ph, and Bn.

TABLE 6

| | | |
|---|---|---|
| 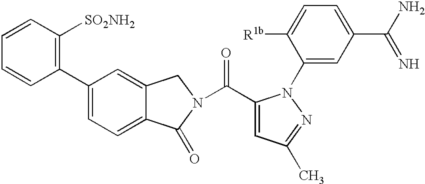 | 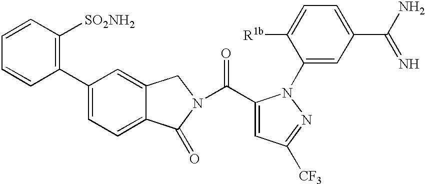 | 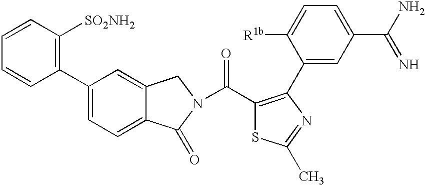 |
| 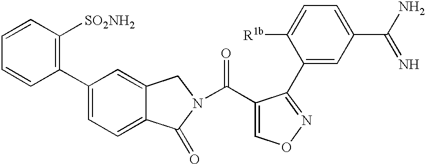 | 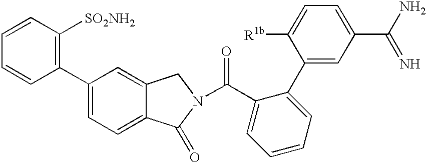 | 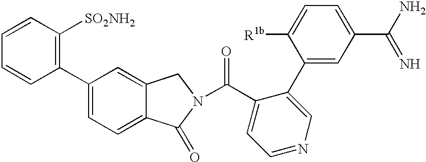 |
| 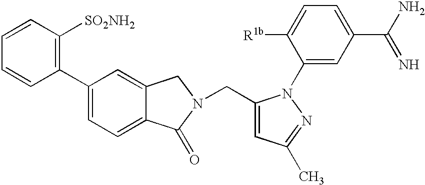 | 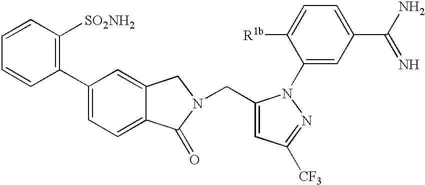 | 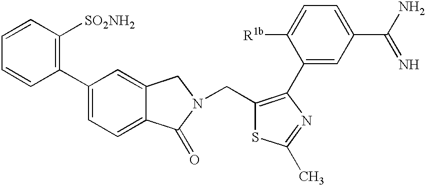 |
| 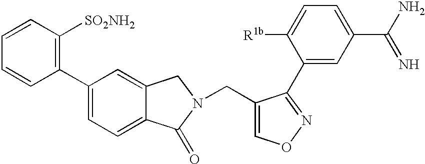 | 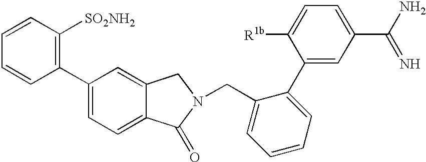 | 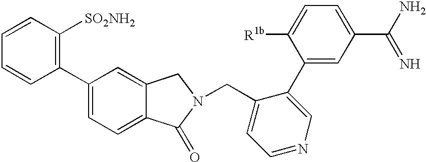 |

For the compounds of Table 6, R^{1b} is selected from H, F, Cl, Br, I, Me, OH, OMe, OPh, OBn, NH₂, OCH₂CH₂NH₂, OCH₂CH₂OH, CONH₂, NO₂, SO₂Me, SO₂NH₂, CN, CH₂OH, CH₂NH₂, CO₂H, CO₂Me, CH₂CO₂H, CH₂CO₂Me, CONMe₂ and CH₂CONMe₂.

TABLE 7

| | | |
|---|---|---|
| 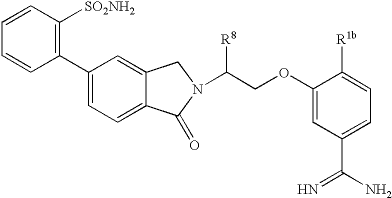 | 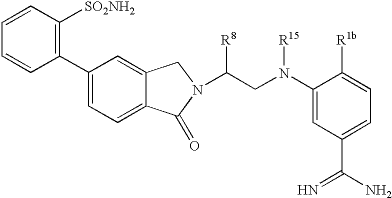 | 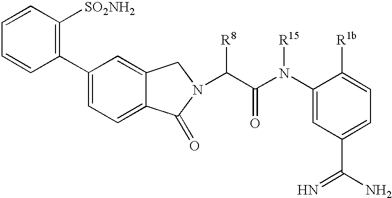 |

For the compounds of Table 7, R^{1b} is selected from H, OH, NH₂, NO₂, F, SO₂Me, CN, CONH₂ and SO₂NH₂; R⁸ is selected from H, Me, Et, Ph, Bn, CO₂H and CO₂Me; and R¹⁵ is selected from H, Me, Et and Bn.

This invention also encompasses all pharmaceutically acceptable isomers, salts, hydrates and solvates of the compounds of formula I. In addition, the compounds of formula I can exist in various isomeric and tautomeric forms, and all such forms are meant to be included in the invention, along with pharmaceutically acceptable salts, hydrates and solvates of such isomers and tautomers.

The compounds of this invention can be isolated as the free acid or base or converted to salts of various inorganic and organic acids and bases. Such salts are within the scope of this invention. Non-toxic and physiologically compatible salts are particularly useful although other less desirable salts may have use in the processes of isolation and purification.

A number of methods are useful for the preparation of the salts described above and are known to those skilled in the art. For example, the free acid or free base form of a compound of one of the formulas above can be reacted with one or more molar equivalents of the desired acid or base in a solvent or solvent mixture in which the salt is insoluble, or in a solvent like water after which the solvent is removed by evaporation, distillation or freeze drying. Alternatively, the free acid or base form of the product can be passed over an ion exchange resin to form the desired salt or one salt form of the product can be converted to another using the same general process.

Prodrug Derivatives of Compounds

This invention also encompasses prodrug derivatives of the compounds contained herein. The term “prodrug” refers to a pharmacologically inactive derivative of a parent drug molecule that requires biotransformation, either spontaneous or enzymatic, within the organism to release the active drug. Prodrugs are variations or derivatives of the compounds of this invention which have groups cleavable under metabolic conditions. Prodrugs become the compounds of the invention which are pharmaceutically active in vivo, when they undergo solvolysis under physiological conditions or undergo enzymatic degradation. Prodrug compounds of this invention can be called single, double, triple etc., depending on the number of biotransformation steps required to release the active drug within the organism, and indicating the number of functionalities present in a precursor-type form. Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include acid derivatives well known to practitioners of the art, such as, for example, esters prepared by reaction of the parent acids with a suitable alcohol, or amides prepared by reaction of the parent acid compound with an amine, or basic groups reacted to form an acylated base derivative. Moreover, the prodrug derivatives of this invention can be combined with other features herein taught to enhance bioavailability.

As mentioned above, the compounds of this invention find utility as therapeutic agents for disease states in mammals which have disorders of coagulation such as in the treatment or prevention of unstable angina, refractory angina, myocardial infarction, transient ischemic attacks, thrombotic stroke, embolic stroke, disseminated intravascular coagulation including the treatment of septic shock, deep venous thrombosis in the prevention of pulmonary embolism or the treatment of reocclusion or restenosis of reperfused coronary arteries. Further, these compounds are useful for the treatment or prophylaxis of those diseases which involve the production and/or action of factor Xa/prothrombinase complex. This includes a number of thrombotic and prothrombotic states in which the coagulation cascade is activated which include but are not limited to, deep venous thrombosis, pulmonary embolism, myocardial infarction, stroke, thromboembolic complications of surgery and peripheral arterial occlusion.

Accordingly, a method for preventing or treating a condition in a mammal characterized by undesired thrombosis comprises administering to the mammal a therapeutically effective amount of a compound of this invention. In addition to the disease states noted above, other diseases, treatable or preventable by the administration of compounds of this invention include, without limitation, occlusive coronary thrombus formation resulting from either thrombolytic therapy or percutaneous transluminal coronary angioplasty, thrombus formation in the venous vasculature, disseminated intravascular coagulopathy, a condition wherein there is rapid consumption of coagulation factors and systemic coagulation which results in the formation of life-threatening thrombi occurring throughout the microvasculature leading to widespread organ failure, hemorrhagic stroke, renal dialysis, blood oxygenation, and cardiac catheterization.

The compounds of the invention also find utility in a method for inhibiting the coagulation biological samples, which comprises the administration of a compound of the invention.

The compounds of the present invention may also be used in combination with other therapeutic or diagnostic agents. In certain preferred embodiments, the compounds of this invention can be coadministered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, aspirin, or warfarin. The compounds of the present invention may act in a synergistic fashion to prevent reocclusion following a successful thrombolytic therapy and/or reduce the time to reperfusion. These compounds may also allow for reduced doses of the thrombolytic agents to be used and therefore minimize potential hemorrhagic side-effects. The compounds of this invention can be utilized in vivo, ordinarily in mammals such as primates, (e.g. humans), sheep, horses, cattle, pigs, dogs, cats, rats and mice, or in vitro.

The biological properties of the compounds of the present invention can be readily characterized by methods that are well known in the art, for example by the in vitro protease activity assays and in vivo studies to evaluate antithrombotic efficacy, and effects on hemostasis and hematological parameters, such as are illustrated in the examples.

Diagnostic applications of the compounds of this invention will typically utilize formulations in the form of solutions or suspensions. In the management of thrombotic disorders the compounds of this invention can be utilized in compositions such as tablets, capsules or elixirs for oral administration, suppositories, sterile solutions or suspensions or injectable administration, and the like, or incorporated into shaped articles. Subjects in need of treatment (typically mammalian) using the compounds of this invention can be administered dosages that will provide optimal efficacy. The dose and method of administration will vary from subject to subject and be dependent upon such factors as the type of mammal being treated, its sex, weight, diet, concurrent medication, overall clinical condition, the particular compounds employed, the specific use for which these compounds are employed, and other factors which those skilled in the medical arts will recognize.

Formulations of the compounds of this invention are prepared for storage or administration by mixing the compound having a desired degree of purity with physiologically acceptable carriers, excipients, stabilizers etc., and can be provided in sustained release or timed release formulations. Acceptable carriers or diluents for therapeutic use are well known in the pharmaceutical field, and are described, for example, in Remington's Pharmaceutical Sciences, Mack Publishing Co., (A. R. Gennaro edit. 1985). Such materials are nontoxic to the recipients at the dosages and concentrations employed, and include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium and/or nonionic surfactants such as Tween, Plurorics or polyethyleneglycol.

Dosage formulations of the compounds of this invention to be used for therapeutic administration must be sterile. Sterility is readily accomplished by filtration through sterile membranes such as 0.2 micron membranes, or by other conventional methods. Formulations typically will be stored in lyophilized form or as an aqueous solution. The pH of the preparations of this invention typically will be 3-11, more preferably 5-9 and most preferably 7-8. It will be understood that use of certain of the foregoing excipients, carriers, or stabilizers will result in the formation of cyclic polypeptide salts. While the preferred route of administration is by injection, other methods of administration are also anticipated such as orally, intravenously (bolus and/or infusion), subcutaneously, intramuscularly, colonically, rectally, nasally, transdermally or intraperitoneally, employing a variety of dosage forms such as suppositories, implanted pellets or small cylinders, aerosols, oral dosage formulations and topical formulations such as ointments, drops and dermal patches. The compounds of this invention are desirably incorporated into shaped articles such as implants which may employ inert materials such as biodegradable polymers or synthetic silicones, for example, Silastic, silicone rubber or other polymers commercially available.

The compounds of the invention may also be administered in the form of liposome delivery systems, such as small unilamellar vesicles, large unilamellar vesicles and multilamellar vesicles. Liposomes can be formed from a variety of lipids, such as cholesterol, stearylamine or phosphatidylcholines.

The compounds of this invention may also be delivered by the use of antibodies, antibody fragments, growth factors, hormones, or other targeting moieties, to which the compound molecules are coupled. The compounds of this invention may also be coupled with suitable polymers as targetable drug carriers. Such polymers can include polyvinylpyrrolidinone, pyran copolymer, polyhydroxy-propyl-methacrylamide-phenol, polyhydroxyethyl-aspartamide-phenol, or polyethyleneoxide-polylysine substituted with palmitoyl residues. Furthermore, compounds of the invention can be coupled to a class of biodegradable polymers useful in achieving controlled release of a drug, for example polylactic acid, polyglycolic acid., copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices can be formed into shaped articles, such as valves, stents, tubing, prostheses and the like.

Therapeutic compound liquid formulations generally are placed into a container having a sterile access port, for example, an intravenous solution bag or vial having a stopper pierceable by hypodermic injection needle.

Therapeutically effective dosages can be determined by either in vitro or in vivo methods. For each particular compound of the present invention, individual determinations can be made to determine the optimal dosage required. The range of therapeutically effective dosages will be influenced by the route of administration, the therapeutic objectives and the condition of the patient. For injection by hypodermic needle, it can be assumed the dosage is delivered into the body's fluids. For other routes of administration, the absorption efficiency must be individually determined for each compound by methods well known in pharmacology. Accordingly, it may be necessary for the therapist to titer the dosage and modify the route of administration as required to obtain the optimal therapeutic effect. The determination of effective dosage levels, that is, the dosage levels necessary to achieve the desired result, will be readily determined by one skilled in the art. Typically, applications of compound are commenced at lower dosage levels, with dosage levels being increased until the desired effect is achieved.

The compounds of the invention can be administered orally or parenterally in an effective amount within the dosage range of about 0.1 to 100 mg/kg, preferably about 0.5 to 50 mg/kg and more preferably about 1 to 20 mg/kg on a regimen in a single or 2 to 4 divided daily doses and/or continuous infusion.

Typically, about 5 to 500 mg of a compound or mixture of compounds of this invention, as the free acid or base form or as a pharmaceutically acceptable salt, is compounded with a physiologically acceptable vehicle, carrier, excipient, binder, preservative, stabilizer, dye, flavor etc., as called for by accepted pharmaceutical practice. The amount of active ingredient in these compositions is such that a suitable dosage in the range indicated is obtained.

Typical adjuvants which can be incorporated into tablets, capsules and the like are binders such as acacia, corn starch or gelatin, and excipients such as microcrystalline cellulose, disintegrating agents like corn starch or alginic acid, lubricants such as magnesium stearate, sweetening agents such as sucrose or lactose, or flavoring agents. When a dosage form is a capsule, in addition to the above materials it may also contain liquid carriers such as water, saline, or a fatty oil. Other materials of various types can be used as coatings or as modifiers of the physical form of the dosage unit. Sterile compositions for injection can be formulated according to conventional pharmaceutical practice. For example, dissolution or suspension of the active compound in a vehicle such as an oil or a synthetic fatty vehicle like ethyl oleate, or into a liposome may be desired. Buffers, preservatives, antioxidants and the like can be incorporated according to accepted pharmaceutical practice.

Preparation of Compounds

The compounds of the present invention can be synthesized by either solid or liquid phase methods described and referenced in standard textbooks, or by a combination of both methods. These methods are well known in the art. See, Bodanszky, “The Principles of Peptide Synthesis”, Hafner, et al., Eds., Springer-Verlag, Berlin, 1984.

Starting materials used in any of these methods are commercially available from chemical vendors such as Aldrich, Sigma, Nova Biochemicals, Bachem Biosciences, and the like, or can be readily synthesized by known procedures.

Reactions are carried out in standard laboratory glassware and reaction vessels under reaction conditions of standard temperature and pressure, except where otherwise indicated.

During the synthesis of these compounds, the functional groups of the amino acid derivatives used in these methods are protected by blocking groups to prevent cross reaction during the coupling procedure. Examples of suitable blocking groups and their use are described in “The Peptides: Analysis, Synthesis, Biology”, Academic Press, Vol. 3 (Gross, et al., Eds., 1981) and Vol. 9 (1987), the disclosures of which are incorporated herein by reference.

Non-limiting exemplary synthesis schemes are outlined directly below, and specific steps are described in the Examples. The reaction products are isolated and purified by conventional methods, typically by solvent extraction into a compatible solvent. The products can be further purified by column chromatography or other appropriate methods.

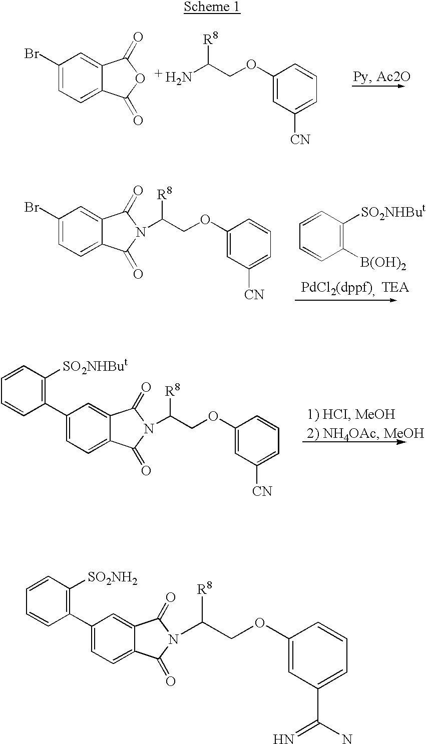

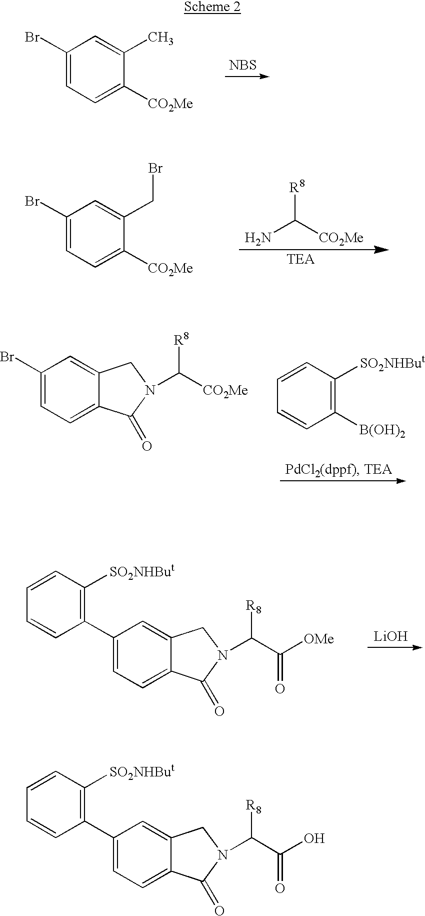

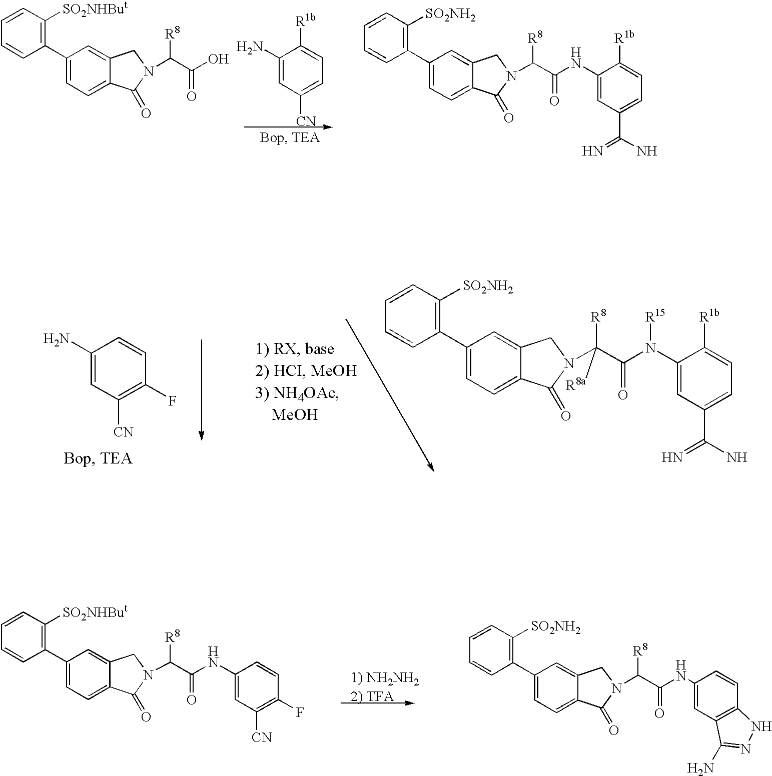

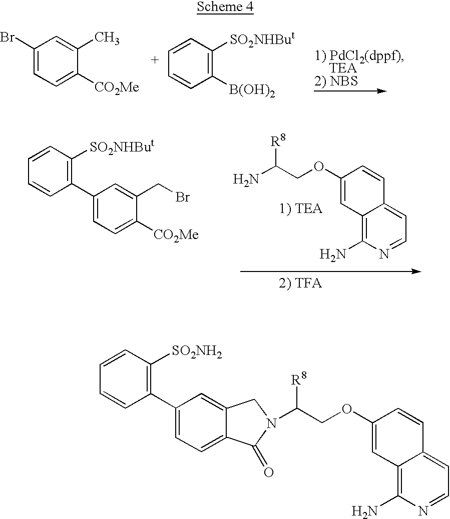

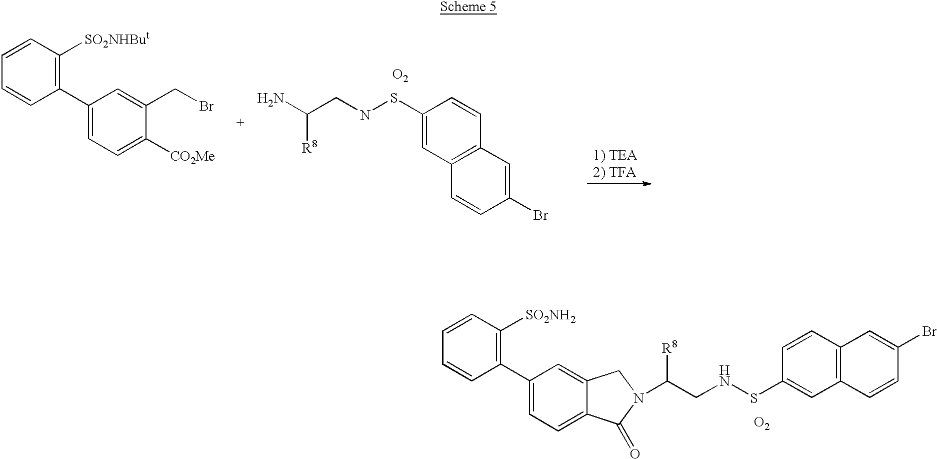

Compositions and Formulations

The compounds of this invention can be isolated as the free acid or base or converted to salts of various inorganic and organic acids and bases. Such salts are within the scope of this invention. Non-toxic ; and physiologically compatible salts are particularly useful although other less desirable salts may have use in the processes of isolation and purification.

A number of methods are useful for the preparation of the salts described above and are known to those skilled in the art. For example, reaction of the free acid or free base form of a compound of the structures recited above with one or more molar equivalents of the desired acid or base in a solvent or solvent mixture in which the salt is insoluble, or in a solvent like water after which the solvent is removed by evaporation, distillation or freeze drying. Alternatively, the free acid or base form of the product can be passed over an ion exchange resin to form the desired salt or one salt form of the product can be converted to another using the same general process.

Diagnostic applications of the compounds of this invention will typically utilize formulations such as solution or suspension. In the management of thrombotic disorders the compounds of this invention can be utilized in compositions such as tablets, capsules or elixirs for oral administration, suppositories, sterile solutions or suspensions or injectable administration, and the like, or incorporated into shaped articles. Subjects in need of treatment (typically mammalian) using the compounds of this invention can be administered dosages that will provide optimal efficacy. The dose and method of administration will vary from subject to subject and be dependent upon such factors as the type of mammal being treated, its sex, weight, diet, concurrent medication, overall clinical condition, the particular compounds employed, the specific use for which these compounds are employed, and other factors which those skilled in the medical arts will recognize.

Formulations of the compounds of this invention are prepared for storage or administration by mixing the compound having a desired degree of purity with physiologically acceptable carriers, excipients, stabilizers etc., and can be provided in sustained release or timed release formulations. Acceptable carriers or diluents for therapeutic use are well known in the pharmaceutical field, and are described, for example, in Remington's Pharmaceutical Sciences, Mack Publishing Co., (A. R. Gennaro edit. 1985). Such materials are nontoxic to the recipients at the dosages and concentrations employed, and include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinalpyrrolidinone, amino acids such as glycine, glutamic acid, aspartic acid, or arginine, monosaccharides, disaccharides, and other carbohydrates including cellulose or its derivatives, glucose, mannose or dextrins, chelatingagents such as EDTA, sugar alcohols such as mannitol or sorbitol, counterions such as sodium and/or nonionic surfactants such as Tween, Pluronics or polyethyleneglycol.

Dosage formulations of the compounds of this invention to be used for therapeutic administration must be sterile. Sterility is readily accomplished by filtration through sterile membranes such as 0.2 micron membranes, or by other conventional methods. Formulations typically will be stored in lyophilized form or as an aqueous solution. The pH of the preparations of this invention typically will be between 3 and 11, more preferably from 5 to 9 and most preferably from 7 to 8. It will be understood that use of certain of the foregoing excipients, carriers, or stabilizers will result in the formation of cyclic polypeptide salts. While the preferred route of administration is by injection, other methods of administration are also anticipated such as intravenously (bolus and/or infusion), subcutaneously, intramuscularly, colonically, rectally, nasally or intraperitoneally, employing a variety of dosage forms such as suppositories, implanted pellets or small cylinders, aerosols, oral dosage formulations and topical formulations such as ointments, drops and dermal patches. The compounds of this invention are desirably incorporated into shaped articles such as implants which may employ inert materials such as biodegradable polymers or synthetic silicones, for example, Silastic, silicone rubber or other polymers commercially available.

The compounds of this invention may also be administered in the form of liposome delivery systems, such as small unilamellar vesicles, large unilamellar vesicles and multilamellar vesicles. Liposomes can be formed from a variety of lipids, such as cholesterol, stearylamine or phosphatidylcholines.

The compounds of this invention may also be delivered by the use of antibodies, antibody fragments, growth factors, hormones, or other targeting moieties, to which the compound molecules are coupled. The compounds of this invention may also be coupled with suitable polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the factor Xa inhibitors of this invention can be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices can be formed into shaped articles, such as valves, stents, tubing, prostheses and the like.

Therapeutic compound liquid formulations generally are placed into a container having a sterile access port, for example, an intravenous solution bag or vial having a stopper pierceable by hypodermic injection needle.

Therapeutically effective dosages can be determined by either in vitro or in vivo methods. For each particular compound of the present invention, individual determinations can be made to determine the optimal dosage required. The range of therapeutically effective dosages will naturally be influenced by the route of administration, the therapeutic objectives, and the condition of the patient. For injection by hypodermic needle, it may be assumed the dosage is delivered into the body's fluids. For other routes of administration, the absorption efficiency must be individually determined for each inhibitor by methods well known in pharmacology. Accordingly, it may be necessary for the therapist to titer the dosage and modify the route of administration as required to obtain the optimal therapeutic effect. The determination of effective dosage levels, that is, the dosage levels necessary to achieve the desired result, will be within the ambit of one skilled in the art. Typically, applications of compound are commenced at lower dosage levels, with dosage levels being increased until the desired effect is achieved.

A typical dosage might range from about 0.001 mg/kg to about 1000 mg/kg, preferably from about 0.01 mg/kg to about 100 mg/kg, and more preferably from about 0.10 mg/kg to about 20 mg/kg. Advantageously, the compounds of this invention can be administered several times daily, and other dosage regimens may also be useful.

Typically, about 0.5 to 500 mg of a compound or mixture of compounds of this invention, as the free acid or base form or as a pharmaceutically acceptable salt, is compounded with a physiologically acceptable vehicle, carrier, excipient, binder, preservative, stabilizer, dye, flavor etc., as called for by accepted pharmaceutical practice. The amount of active ingredient in these compositions is such that a suitable dosage in the range indicated is obtained.

Typical adjuvants which can be incorporated into tablets, capsules and the like are a binder such as acacia, corn starch or gelatin, and excipient such as microcrystalline cellulose, a disintegrating agent like corn starch or alginic acid, a lubricant such as magnesium stearate, a sweetening agent such as sucrose or lactose, or a flavoring agent. When a dosage form is a capsule, in addition to the above materials it may also contain a liquid carrier such as water, saline, a fatty oil. Other materials of various types can be used as coatings or as modifiers of the physical form of the dosage unit. Sterile compositions for injection can be formulated according to conventional pharmaceutical practice. For example, dissolution or suspension of the active compound in a vehicle such as an oil or a synthetic fatty vehicle like ethyl oleate, or into a liposome may be desired. Buffers, preservatives, antioxidants and the like can be incorporated according to accepted pharmaceutical practice.

In practicing the methods of this invention, the compounds of this invention can be used alone or in combination, or in combination with other therapeutic or diagnostic agents. In certain preferred embodiments, the compounds of this inventions can be coadministered along with other compounds typically prescribed for these conditions according to generally accepted medical practice, such as anticoagulant agents, thrombolytic agents, or other antithrombotics, including platelet aggregation inhibitors, tissue plasminogen activators, urokinase, prourokinase, streptokinase, heparin, aspirin, or warfarin. The compounds of this invention can be utilized in vivo, ordinarily in mammals such as primates, such as humans, sheep, horses, cattle, pigs, dogs, cats, rats and mice, or in vitro.

The preferred compounds of the present invention are characterized by their ability to inhibit thrombus formation with acceptable effects on classical measures of coagulation parameters, platelets and platelet function, and acceptable levels of bleeding complications associated with their use. Conditions characterized by undesired thrombosis would include those involving the arterial and venous vasculature.

With respect to the coronary arterial vasculature, abnormal thrombus formation characterizes the rupture of an established atherosclerotic plaque which is the major cause of acute myocardial infarction and unstable angina, as well as also characterizing the occlusive coronary thrombus formation resulting from either thrombolytic therapy or percutaneous transluminal coronary angioplasty (PTCA).

With respect to the venous vasculature, abnormal thrombus formation characterizes the condition observed in patients undergoing major surgery in the lower extremities or the abdominal area who often suffer from thrombus formation in the venous vasculature resulting in reduced blood flow to the affected extremity and a predisposition to pulmonary embolism. Abnormal thrombus formation further characterizes disseminated intravascular coagulopathy commonly occurs within both vascular systems during septic shock, certain viral infections and cancer, a condition wherein there is rapid consumption of coagulation factors and systemic coagulation which results in the formation of life-threatening thrombi occurring throughout the microvasculature leading to widespread organ failure.

The compounds of this present invention, selected and used as disclosed herein, are believed to be useful for preventing or treating a condition characterized by undesired thrombosis, such as (a) the treatment or prevention of any thrombotically mediated acute coronary syndrome including myocardial infarction, unstable angina, refractory angina, occlusive coronary thrombus occurring post-thrombolytic therapy or post-coronary angioplasty, (b) the treatment or prevention of any thrombotically mediated cerebrovascular syndrome including embolic stroke, thrombotic stroke or transient ischemic attacks, (c) the treatment or prevention of any thrombotic syndrome occurring in the venous system including deep venous thrombosis or pulmonary embolus occurring either spontaneously or in the setting of malignancy, surgery or trauma, (d) the treatment or prevention of any coagulopathy including disseminated intravascular coagulation (including the setting of septic shock or other infection, surgery, pregnancy, trauma or malignancy and whether associated with multi-organ failure or not), thrombotic thrombocytopenic purpura, thromboangiitis obliterans, or thrombotic disease associated with heparin induced thrombocytopenia, (e) the treatment or prevention of thrombotic complications associated with extracorporeal circulation (e.g. renal dialysis, cardiopulmonary bypass or other oxygenation procedure, plasmapheresis), (f) the treatment or prevention of thrombotic complications associated with instrumentation (e.g. cardiac or other intravascular catheterization, intra-aortic balloon pump, coronary stent or cardiac valve), and (g) those involved with the fitting of prosthetic devices.

Anticoagulant therapy is also useful to prevent coagulation of stored whole blood and to prevent coagulation in other biological samples for testing or storage. Thus the compounds of this invention can be added to or contacted with any medium containing or suspected to contain factor Xa and in which it is desired that blood coagulation be inhibited, e.g., when contacting the mammal's blood with material such as vascular grafts, stents, orthopedic prostheses, cardiac stents, valves and prostheses, extra corporeal circulation systems and the like.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### EXAMPLES

### Examples 1 and 2

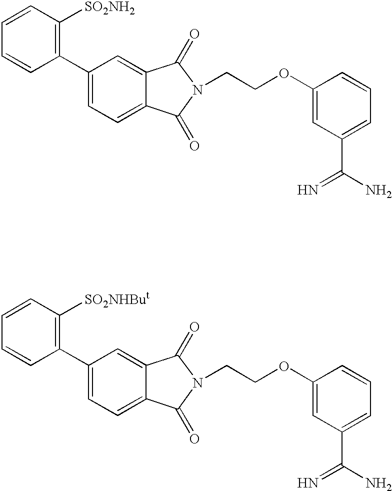

Step 1: To a solution of 3-(2-aminoethoxy)benzonitrile (276 mg, 1 mmol, 1.0 equiv) in 5 mL of methanol at room temperature was added TEA (630 μL, 4.5 equiv) and 4-bromophthalic anhydride (227 mg, 1.0 equiv). After stirring at room temperature for 3 h, the solvent was evaporated and the residue was vacuum dried. The residue in 10 mL of pyridine was then treated with 12 mL of acetic anhydride at room temperature overnight. The volatile solvent was evaporated, and the residue was taken up with EtOAc, washed with H₂O, dried over MgSO₄ and column purified with 1:3 EtOAc/hexanes on silica gel to give 3-[2-(5-bromo-1,3-dioxoisoindolin-2-yl)ethoxy]benzonitrile in 84% yield. LRMS found for C₁₇H₁₂BrN₂O₃ (M+H)⁺: 371.05.

Step 2: A solution of 3-[2-(5-bromo-1,3-dioxoisoindolin-2-yl)ethoxy]benzonitrile (124 mg, 0.3 mmol, 1.0 equiv), 2-tert-butylaminosulfonyl phenyl boronic acid (86 mg, 1.0 equiv), PdCl₂(dppf) (27 mg, 0.1 equiv), TEA (230 μL, 5.0 equiv) in 5 mL of DME was degassed with Ar for 15 min, refluxed overnight. After cooling to room temperature, the mixture was diluted with EtOAc, washed with water, dried over MgSO₄, and evaporated. Flash chromatography on silica gel gave 3-{2-[5-(2′-tert-butylaminosulfonyl)phenyl-1,3-dioxoisoindolin-2-yl]ethoxy}benzonitrile in 75% yield. LRMS found for C₂₇H₂₆N₃O₅S (M+H)⁺: 504.16.

Step 3: 3-{2-[5-(2′-tert-butylaminosulfonyl)phenyl-1,3-dioxoisoindolin-2-yl]ethoxy}benzonitrile (90 mg) was dissolved in 5 mL of methanol. The reaction mixture was cooled to 0° C. and HCl gas was bubbled in until saturation. The mixture was stirred at room temperature overnight. The solvent was evaporated and the resulting residue was treated with ammonium acetate (100 mg) and 10 ml methanol at reflux temperature for 2 h. The solvent was removed at reduced pressure and the crude benzamidine was purified by HPLC (C18 reversed phase) eluting with 0.5% TFA in H₂O/CH₃CN to give 3-{2-[5-(2′-aminosulfonyl)phenyl-1,3-dioxoisoindolin-2-yl]ethoxy}benzamidine (31%), LRMS found for C₂₃H₂₁N₄O₅S (M+H)⁺: 465.10; and 3-{2-[5-(2′-tert-butylaminosulfonyl)phenyl-1,3-dioxoisoindolin-2-yl]ethoxy}benzamidine (27 mg, 34%). LRMS found for C₂₃H₂₁N₄O₅S (M+H)⁺: 521.25.

### Example 3

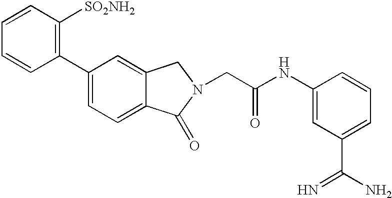

Step 1: A mixture of 4-bromo-2-methylbenzoic acid (12 g) and PTSA (1 g) in 100 mL of methanol was refluxed overnight. After cooling to room temperature, methanol was evaporated and the residue was diluted with EtOAc, washed with saturated NaHCO₃. The organic layer was dried over MgSO₄, filtered and evaporated. The product (99%) was used in the next step without further purification.

Step 2: Methyl 4-bromo-2-methylbenzoate (4.58 g, 20 mmol, 1 equiv) and NBS (3.56 g, 1 equiv) in 50 mL of CCl₄ was treated with benzoyl peroxide (240 mg, 0.05 equiv) at reflux for 6 h. After cooling to room temperature, the insoluble material was filtered off and the filtrate was evaporated to give methyl 4-bromo-2-bromomethylbenzoate, which was used directly in the next step without further purification.

Step 3: Methyl 4-bromo-2-bromomethylbenzoate (1.54 g, ˜60% pure, 5 mmol, 1 equiv), ethyl glycinate hydrochloride (698 mg, 1 equiv) and 1.4 mL of TEA in 30 mL of benzene was refluxed for 3 h and then cooled to rt. After benzene was evaporated, the residue was diluted with EtOAc, washed with water and brine. Flash chromatography on silica gel with 1:3 EtOAc/hexanes gave ethyl 2-(5-bromo-1-oxoisoindolin-2-yl) acetate in 73% yield. LRMS found for C₁₂H₁₃BrNO₃ (M+H)⁺: 298.11.

Step 4: A solution of ethyl 2-(5-bromo-1-oxoisoindolin-2-yl) acetate (370 mg, 1.24 mmol, 1.0 equiv), 2-tert-butylaminosulfonyl phenyl boronic acid (319 mg, 1.0 equiv), PdCl₂(dppf) (101 mg, 0.1 equiv), TEA (860 μL, 5.0 equiv) in 10 mL of DME was degassed with Ar for 15 min, then heated to reflux overnight. After cooling to room temperature, the mixture was diluted with EtOAc, washed with water, dried over MgSO₄, evaporated. Flash chromatography on silica gel with 1:1 EtOAc/hexanes gave ethyl 2-[5-(2′-tert-butylaminosulfonyl)phenyl-oxoiscindolin-2-yl]acetate in 89% yield. LRMS found for C₂₂H₂₇N₂O₅S (M+H)⁺: 431.15.

Step 5: A solution of ethyl 2-[5-(2′-tert-butylaminosulfonyl)phenyl-oxoisoindolin-2-yl]acetate (420 mg, 1.0 mmol, 1.0 equiv) in 5 mL of THF was treated with 2.1 mL of 1N LiOH at 0° C. for 1 h. THF was evaporated and the aqueous residue was acidified with 1N HCl, extracted with EtOAc. The organic layer was dried over MgSO₄, filtered and evaporated to give 2-[5-(2′-tert-butylaminosulfonyl)phenyl-oxoisoindolin-2-yl]acetic acid (99%). LRMS found for C₂₀H₂₂N₂O₅S (M+H)⁺: 402.13.

Step 6: A solution of 2-[5-(2′-tert-butylaminosulfonyl)phenyl-oxoisoindolin-2-yl]acetic acid (200 mg, 0.5 mmol, 1.0 equiv), Bop (443 mg, 2 equiv) in 5 mL of DMF was treated with 700 μL of TEA for 15 min at 0° C. and 3-aminobenzonitrile (118 mg, 2 equiv) was added and stirred at room temperature overnight. The solution was then diluted with EtOAc washed with water and dried over MgSO₄. Flash chromatography over silica gel with 1:1 EtOAc/hexanes gave 2-[5-(2′-tert-butylaminosulfonyl)phenyl-oxoisoindolin-2-yl]-N-(3-cyanophenyl)acetamide (72%). LRMS found for C₂₇H₂₇N₄O₄S (M+H)⁺: 503.20.

Step 7: A solution of 2-[5-(2′-tert-butylaminosulfonyl)phenyl-oxoisoindolin-2-yl]-N-(3-cyanophenyl)acetamide (60 mg, 0.12 mmol, 1.0 equiv) and TEA (170 μL, 10 equiv) in 5 mL of EtOH was treated with hydroxylamine hydrochloride (45 mg, 5 equiv) overnight. EtOH was evaporated and the residue was stirred in 1.5 mL of Ac₂O at room temperature for 2 h. The mixture was diluted with 3 mL of EtOH, 20 mg of 10% Pd/C was added and stirred under 1 atm H₂ for overnight. After filtration and evaporation, HPLC gave 2-[5-(2′-aminosulfonyl)phenyl-oxoisoindolin-2-yl]-N-(3-amidinophenyl)acetamide in 67% yield. LRMS found for C₂₃H₂₁N₅O₄S (M+H)⁺: 463.15.

### Example 4

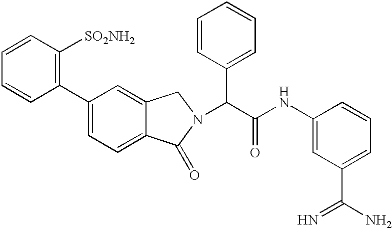

Step 1: Methyl 4-bromo-2-bromomethylbenzoate (1.22 g, ˜60% pure, 3.96 mmol, 1 equiv), methyl phenylglycinate hydrochloride (800 mg, 1 equiv) and 1.7 mL of TEA in 40 mL of toluene was refluxed for overnight and then cooled to rt. After toluene was evaporated, the residue was diluted with EtOAc, washed with water and saturated brine. Flash chromatography on silica gel with 1:3 EtOAc/hexanes gave methyl 2-(5-bromo-1-oxoisoindolin-2-yl)-2-phenyl acetate in 81% yield. LRMS found for C₁₇H₁₅BrNO₃ (M+H)⁺: 360.10.

Step 2: A solution of methyl 2-(5-bromo-1-oxoisoindolin-2-yl)-2-phenyl acetate (540 mg, 1.5 mmol, 1.0 equiv), 2-t-butylaminosulfonyl phenyl boronic acid (385 mg, 1.0 equiv), PdCl₂(dppf) (122 mg, 0.1 equiv), TEA (1.1 mL, 5.0 equiv) in 10 mL of DME was degassed with Ar for 15 min, then heated to reflux overnight. After cooling to room temperature, the mixture was diluted with EtOAc, washed with water, dried over MgSO₄, evaporated. Flash chromatography on silica gel with 1:1 EtOAc/hexanes gave methyl 2-[5-(2′-tert-butylaminosulfonyl)phenyl-oxoisoindolin-2-yl]-2-phenylacetate in 92% yield. LRMS found for C₂₇H₂₉N₂O₅S (M+H)⁺: 493.17.

Step 3: A solution of methyl 2-[5-(2′-tert-butylaminosulfonyl)phenyl-oxoisoindolin-2-yl]-2-phenylacetate (520 mg, 1.06 mmol, 1.0 equiv) in 5 mL of THF was treated with 2.2 mL of 1N LiOH at 0° C. for 1 h. THF was evaporated and the aqueous residue was acidified with 1N HCl, extracted with EtOAc. The organic layer was dried over MgSO₄, filtered and evaporated to give 2-[5-(2′-tert-butylaminosulfonyl)phenyl-oxoisoindolin-2-yl]-2-phenylacetic acid (99%). LRMS found for C₂₆H₂₇N₂O₅S (M+H)⁺: 479.17.

Step 4: A solution of 2-[5-(2′-tert-butylaminosulfonyl)phenyl-oxoisoindolin-2-yl]-2-phenylacetic acid (240 mg, 0.5 mmol, 1.0 equiv), Bop (443 mg, 2 equiv) in 5 mL of DMF was treated with 700 μL of TEA for 15 min at 0° C. and 3-aminobenzonitrile (118 mg, 2 equiv) was added and stirred at room temperature overnight. The solution was then diluted with EtOAc washed with water and dried over MgSO₄. Flash chromatography over silica gel with 1:1 EtOAc/hexanes gave 2-[5-(2′-tert-butylaminosulfonyl)phenyl-oxoisoindolin-2-yl]-N-(3-cyanophenyl)-2-phenylacetamide (75%). LRMS found for C₃₃H₃₁N₄O₄S (M+H)⁺: 579.21.

Step 5: 2-[5-(2′-tert-butylaminosulfonyl)phenyl-oxoisoindolin-2-yl]-N-(3-cyanophenyl)-2-phenylacetamide obtained in step 3 (35 mg) was dissolved in 3 mL of methanol. The reaction mixture was cooled to 0° C. and HCl gas was bubbled in until saturation. The mixture was stirred at room temperature overnight. The solvent was evaporated and the resulting residue was treated with ammonium acetate (100 mg) and 5 ml methanol at reflux temperature for 2 h. The solvent was removed at reduced pressure and the crude benzamidine was purified by HPLC (C18 reversed phase) eluting with 0.5% TFA in H₂O/CH₃CN to give 2-[5-(2′-aminosulfonyl)phenyl-oxoisoindolin-2-yl]-N-(3-amidinophenyl)-2-phenylacetamide in 67% yield. LRMS found for C₂₉H₂₆N₅O₄S (M+H)⁺: 540.17.

### Examples 5 and 6

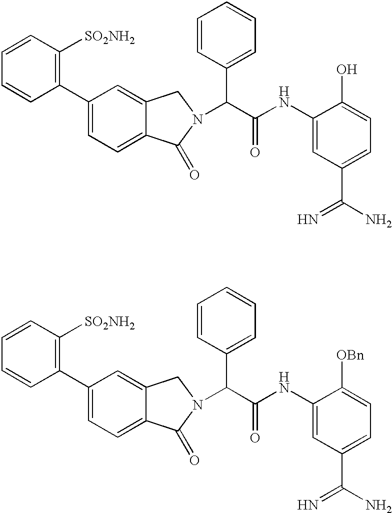

Step 1: A solution of 2-[5-(2′-tert-butylaminosulfonyl)phenyl-oxoisoindolin-2-yl]-2-phenylacetic acid (72 mg, 0.15 mmol, 1.0 equiv), Bop (133 mg, 2.0 equiv) in 3 mL of DMF was treated with 210 μL of TEA for 15 min at 0° C. and 3-amino-4-benzoxybenzonitrile (22 mg, 1.5 equiv) was added and stirred at room temperature overnight. The solution was then diluted with EtOAc washed with water and dried over MgSO4. Flash chromatography over silica gel with 1:1.5 EtOAc/hexanes gave 2-[5-(2′-tert-butylaminosulfonyl)phenyl-oxoisoindolin-2-yl]-N-(2-benzoxy-5-cyano-phenyl)-2-phenylacetamide (84%). LRMS found for C₄₀H₃₇N₄O₅S (M+H)⁺: 685.25.

Step 2: 2-[5-(2′-tert-butylaminosulfonyl)phenyl-oxoisoindolin-2-yl]-N-(2-benzoxy-5-cyano-phenyl)-2-phenylacetamide obtained in step 3 (50 mg) was dissolved in 3 mL of methanol. The reaction mixture was cooled to 0° C. and HCl gas was bubbled in until saturation. The mixture was stirred at room temperature overnight. The solvent was evaporated and the resulting residue was treated with ammonium acetate (100 mg) and 5 ml methanol at reflux temperature for 2 h. The solvent was removed at reduced pressure and the crude benzamidine was purified by HPLC (C18 reversed phase) eluting with 0.5% TFA in H₂O/CH₃CN to give 2-[5-(2′-aminiosulfonyl)phenyl-oxoisoindolin-2-yl]-N-(2-benzoxy-5-amidino-phenyl)-2-phenylacetamide in 35% yield; and 2-[5-(2′-tert-butylaminosulfonyl)phenyl-oxoisoindolin-2-yl]-N-(2-benzoxy-5-amidino-phenyl)-2-phenylacetamide in 37% yield. LRMS found for C₃₆H₃₂N₅O₅S (M+H)⁺: 646.21.

### Example 7

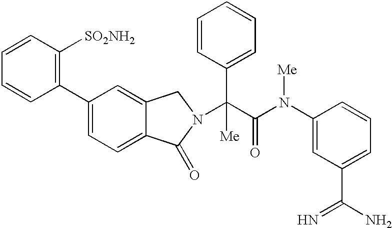

Step 1: A solution of 2-[5-(2′-tert-butylaminosulfonyl)phenyl-oxoisoindolin-2-yl]-N-(3-cyanophenyl)-2-phenylacetamide (57.8 mg, 0.1 mmol, 1.0 equiv), and MeI (20 mL, 3.0 equiv) in 2 mL of DMF was treated with Cs₂CO₃ (325.82 mg, 2 equiv) for 2 h. The solution was then diluted with EtOAc, washed with water and dried over MgSO₄. Flash chromatography over silica gel with 1:1.5 EtOAc/hexanes gave 2-[5-(2′-tert-butylaminosulfonyl)phenyl-oxoisoindolin-2-yl]-N-(3-cyanophenyl)-N-methyl-2-phenylpropanamide in 91% yield. LRMS found for C₃₅H₃₅N₄O₄S (M+H)⁺: 607.25.

Step 2: 2-[5-(2′-tert-butylaminosulfonyl)phenyl-oxoisoindolin-2-yl]-N-(3-cyanophenyl)-N-methyl-2-phenylpropanamide (50 mg) was dissolved in 3 mL of methanol. The reaction mixture was cooled to 0° C. and HCl gas was bubbled in until saturation. The mixture was stirred at room temperature overnight. The solvent was evaporated and the resulting residue was treated with ammonium acetate (100 mg) and 5 ml methanol at reflux temperature for 2 h. The solvent was removed at reduced pressure and the crude benzamidine was purified by HPLC (C18 reversed phase) eluting with 0.5% TFA in H₂O/CH₃CN to give 2-[5-(2′-aminosulfonyl)phenyl-oxoisoindolin-2-yl]-N-(3-amidinophenyl)-N-methyl-2-phenylpropanamide the desired salt in 63% yield. LRMS found for C₃₁H₃₀N₅O₄S (M+H)⁺: 568.21.

### Example 8

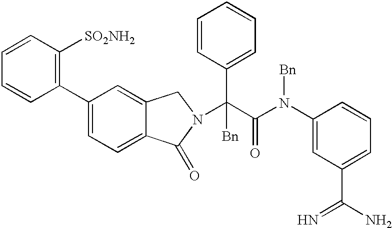

Step 1: A solution of 2-[5-(2′-tert-butylaminosulfonyl)phenyl-oxoisoindolin-2-yl]-N-(3-cyanophenyl)-2-phenylacetamide (57.8 mg, 0.1 mmol, 1.0 equiv), and BnBr (40 μL, 3.0 equiv) in 2 mL of DMF was treated with Cs₂CO₃ (65 mg, 2 equiv) for 2 h. The solution was then diluted with EtOAc, washed with water and dried over MgSO₄. Flash chromatography over silica gel with 1:1.5 EtOAc/hexanes gave 2-[5-(2′-tert-butylaminosulfonyl)phenyl-oxoisoindolin-2-yl]-N-(3-cyanophenyl)-N-methyl-N-benzyl-2,3-diphenylpropanamide in 77% yield. LRMS found for C₄₇H₄₃N₄O₄S (M+H)⁺: 759.31.

Step 2: 2-[5-(2′-tert-butylaminosulfonyl)phenyl-oxoisoindolin-2-yl]-N-(3-cyanophenyl)-N-methyl-N-benzyl-2,3-diphenylpropanamide (50 mg) was dissolved in 3 mL of methanol. The reaction mixture was cooled to 0° C. and HCl gas was bubbled in until saturation. The mixture was stirred at room temperature overnight. The solvent was evaporated and the resulting residue was treated with ammonium acetate (100 mg) and 5 ml methanol at reflux temperature for 2 h. The solvent was removed at reduced pressure and the crude benzamidine was purified by HPLC (C18 reversed phase) eluting with 0.5% TFA in H₂O/CH₃CN to give 2-[5-(2′-aminosulfonyl)phenyl-oxoisoindolin-2-yl]-N-(3-amidinophenyl)-N-methyl-N-benzyl-2,3-diphenylpropanamide the desired salt in 54% yield. LRMS found for C₄₃H₃₈N₅O₄S (M+H)⁺: 720.25.

### Example 9

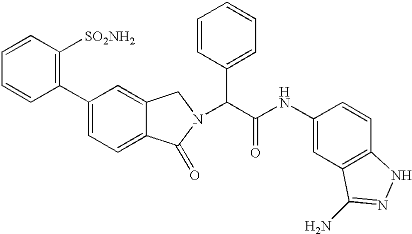

Step 1: A solution of 2-[5-(2′-tert-butylaminosulfonyl)phenyl-oxoisoindolin-2-yl]-2-phenylacetic acid (150 mg, 0.3 mmol, 1.0 equiv), Bop (266 mg, 2.0 equiv) in 3 mL of DMF was treated with 500 μL of TEA for 15 min at 0° C. and 35-amino-2-fluorobenzonitrile (55 mg, 2 equiv) was added and stirred at room temperature overnight. The solution was then diluted with EtOAc washed with water and dried over MgSO₄. Flash chromatography over silica gel with 1:1.5 EtOAc/hexanes gave 2-[5-(2′-tert-butylaminosulfonyl)phenyl-oxoisoindolin-2-yl]-N-(3-cyano-4-fluorophenyl)-2-phenylacetamide (78%). LRMS found for C₃₃H₃₀N₄O₄SF (M+H)⁺: 597.15.

Step 2: A solution of 2-[5-(2′-tert-butylaminosulfonyl)phenyl-oxoisoindolin-2-yl]-N-(3-cyano-4-fluorophenyl)-2-phenylacetamide (50 mg, 0.084 mmol, 1 equiv) and hydrazine (120 μL, 30 equiv) in 3 mL of ethanol was refluxed for 7 days. The solvent was removed at reduced pressure and the residue was vacuum dried and was used in the next step directly.

Step 3: The crude mixture obtained above was treated with 1.5 mL of TFA at reflux for 2 h. After removing the volatile, HPLC (C18 reversed phase) eluting with 0.5% TFA in H₂O/CH₃CN to give the desired product in 58% yield. LRMS found for C₂₉H₂₅N₆O₄S (M+H)⁺: 553.15.

### Example 10

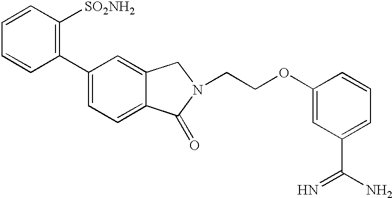

Step 1: A solution of methyl 4-bromo-2-methyl benzoate (370 mg, 1.24 mmol, 1.0 equiv), 2-t-butylaminosulfonyl phenyl boronic acid (319 mg, 1.0 equiv), PdCl₂(dppf) (101 mg, 0.1 equiv), TEA (860 μL, 5.0 equiv) in 10 mL of DME was degassed with Ar for 15 min, then heated to reflux overnight. After cooling to room temperature, the mixture was diluted with EtOAc, washed with water, dried over MgSO₄, evaporated. Flash chromatography on silica gel with 1:1 EtOAc/hexanes gave methyl 4-(2′-tert-butylaminosulfonyl)phenyl-2-methyl benzoate in 86% yield. LRMS found for C₁₉H₂₄NO₄S (M+H)⁺: 362.15.

Step 2: 4-(2′-tert-butylaminosulfonyl)phenyl-2-methyl benzoate (4.58 g, 20 mmol, 1 equiv) and NBS (3.56 g, 1 equiv) in 50 mL of CCl4 was treated with benzoyl peroxide (240 mg, 0.05 equiv) at reflux for 6 h. After cooling to room temperature, the insoluble material was filtered off and the filtrate was evaporated to 4-(2′-tert-butylaminosulfonyl)phenyl-2-bromomethyl benzoate, which was used directly in the next step without further purification.

Step 3: 4-(2′-tert-butylaminosulfonyl)phenyl-2-bromomethyl benzoate (1.22 g, ˜60% pure, 3.96 mmol, 1 equiv), 3-(2-aminoethoxy)benzonitrile (800 mg, 1 equiv) and 1.7 mL of TEA in 40 mL of toluene was refluxed for overnight and then cooled to rt. After toluene was evaporated, the residue was diluted with EtOAc, washed with water and saturated brine Flash chromatography on silica gel. with 1:3 EtOAc/hexanes gave 3-{2-[5-(2′-tert-butylaminosulfonyl)phenyl-1-oxoisoindolin-2-yl]ethoxy}benzonitrile in 45% yield. LRMS found for C₂₇H₂₈N₃O₄S (M+H)⁺: 490.10.

Step 4: 3-{2-[5-(2′-tert-butylaminosulfonyl)phenyl-1-oxoisoindolin-2-yl]ethoxy}benzonitrile (50 mg) was dissolved in 3 mL of methanol. The reaction mixture was cooled to 0° C. and HCl gas was bubbled in until saturation. The mixture was stirred at room temperature overnight. The solvent was evaporated and the resulting residue was treated with ammonium acetate (100 mg) and 5 ml methanol at reflux temperature for 2 h. The solvent was removed at reduced pressure and the crude benzamidine was purified by HPLC (C18 reversed phase) eluting with 0.5% TFA in H₂O/CH₃CN to give 3-{2-[5-(2′-aminosulfonyl)phenyl-1-oxoisoindolin-2-yl]ethoxy}benzoamidine the desired salt in 83% yield. LRMS found for C₂₃H₂₃N₄O₄S (M+H)⁺: 451.10.

### Example 11

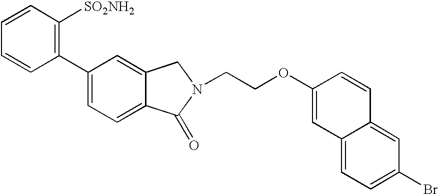

Step 1: 4-(2′-tert-butylaminosulfonyl)phenyl-2-bromomethyl benzoate (1.22 g, ˜60% pure, 3.96 mmol, 1 equiv), 6-bromo-2-(2-amino)ethoxynaphthalene (800 mg, 1 equiv) and 1.7 mL of TEA in 40 mL of toluene was refluxed for overnight and then cooled to rt. After toluene was evaporated, the residue was diluted with EtOAc, washed with water and saturated brine. Flash chromatography on silica gel with 1:1 EtOAc/hexanes gave 2-{2-[5-(2′-tert-butylaminosulfonyl)phenyl-1-oxoisoindolin-2-yl]ethoxy}-6-bromonaphthalene in 66% yield. LRMS found for C₃₀H₃₀BrN2O₄ (M+H)⁺: 561.14.

Step 2: 2-{2-[5-(2′-tert-butylaminosulfonyl)phenyl-1-oxoisoindolin-2-yl]ethoxy}-6-bromonaphthalene was treated with 1.5 mL of TFA at reflux for 2 h. After removing the volatile, HPLC (C18 reversed phase) eluting with 0.5% TFA in H₂O/CH₃CN to give 2-{2-[5-(2′-aminosulfonyl)phenyl-1-oxoisoindolin-2-yl]ethoxy}-6-bromonaphthalene in 81% yield. LRMS found for C₂₆H₂₂BrN₂O₄S (M+H)⁺: 537.05.

### Example 12

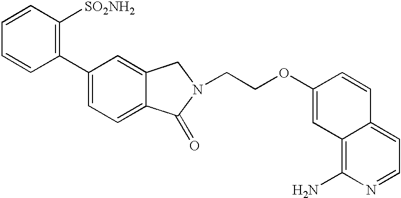

Step 1: 4-(2′-tert-butylaminosulfonyl)phenyl-2-bromomethyl benzoate (150 mg, ˜60% pure, 0.2 mmol, 1 equiv), 1-amino-7-(2-aminoethoxy)isoquinoline (61 mg, 1.5 equiv) and 1.0 mL of TEA in 10 mL of benzene was refluxed for overnight and then cooled to rt. After benzene was evaporated, the residue was diluted with EtOAc, washed with water and saturated brine. The organic layer was dried over MgSO₄, filtered and evaporated.

Step 2: 1-amino-7-{2-[5-(2′-tert-butylaminosulfonyl)phenyl-1-oxoisoindolin-2-yl]ethoxy}isoquinoline obtained above was treated with 1.5 mL of TFA at reflux for 2 h. After removing the volatile, HPLC (C18 reversed phase) eluting with 0.5% TFA in H₂O/CH₃CN to give 1-amino-7-{2-[5-(2′-aminosulfonyl)phenyl-1-oxoisoindolin-2-yl]ethoxy}isoquinoline in 71% yield. LRMS found for C₂₅H₂₃N₄O₄S (M+H)⁺: 475.14.

### Example 13

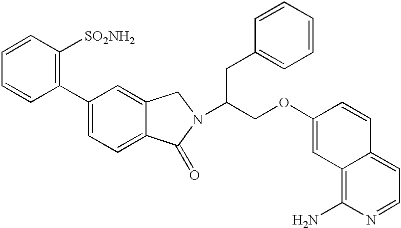

Step 1: 4-(2′-tert-butylaminosulfonyl)phenyl-2-bromomethyl benzoate (150 mg, ˜60% pure, 0.2 mmol, 1 equiv), 1-amino-7-(2-amino-2-benzylethoxy)isoquinoline (88 mg, 1.5 equiv) and 1.0 mL of TEA in 10 mL of benzene was refluxed for overnight and then cooled to rt. After benzene was evaporated, the residue was diluted with EtOAc, washed with water and saturated brine. The organic layer was dried over MgSO₄, filtered and evaporated.

Step 2: 1-amino-7-{2-[5-(2′-tert-butylaminosulfonyl)phenyl-1-oxoisoindolin-2-yl]-2-benzylethoxy}isoquinoline was treated with 1.5 mL of TFA at reflux for 2 h. After removing the volatile, HPLC (C18 reversed phase) eluting with 0.5% TFA in H₂O/CH₃CN to give 1-amino-7-{2-[5-(2′-aminosulfonyl)phenyl-1-oxoisoindolin-2-yl]-2-benzylethoxy}isoquinoline in 79% yield. LRMS found for C₃₂H₂₉N₄O₄S (M+H)⁺: 565.15.

### Example 14

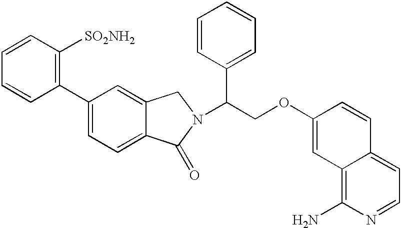

Step 1: 4-(2′-tert-butylaminosulfonyl)phenyl-2-bromomethyl benzoate (150 g, ˜60% pure, 0.2 mmol, 1 equiv), 1-amino-7-(2-amino-2-phenylethoxy)isoquinoline (84 mg, 1 equiv) and 1.0 mL of TEA in 10 mL of benzene was refluxed for overnight and then cooled to rt. After benzene was evaporated, the residue was diluted with EtOAc, washed with water and saturated brine. The organic layer was dried over MgSO₄, filtered and evaporated.

Step 2: 1-amino-7-{2-[5-(2′-tert-butylaminosulfonyl)phenyl-1-oxoisoindolin-2-yl]-2-phenylethoxy}isoquinoline was treated with 1.5 mL of TFA at reflux for 2 h. After removing the volatile, HPLC (C18 reversed phase) eluting with 0.5% TFA in H₂O/CH₃CN to give 1-amino-7-{2-[5-(2′-aminosulfonyl)phenyl-1-oxoisoindolin-2-yl]-2-phenylethoxy}isoquinoline in 69% yield. LRMS found for C₃₁H₂₇N₄O₄S (M+H)⁺: 551.15.

### Example 15

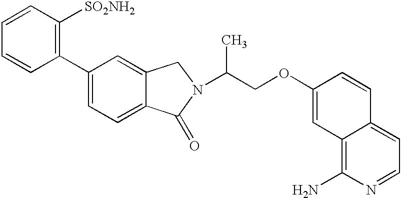

The title compound was prepared in a similar way Example 14 was made. ES-MS (M+H)⁺: 489.20.

### Example 16

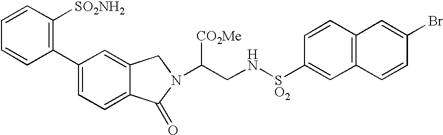

Step 1: A suspension of N-Boc-asparigine (2.32 g, 10 mmol) in a mixture of isopropanol (8 mL), methyl acetate (5 mL) and water (1 mL) was cooled to 5° C. Iodobenzene diacetate (3.65 g, 11 mmol) was added and the mixture was warmed to room temperature and stirred for 2 hours, then slowly heated up to 50° C. over 90 min. Finally, the mixture was cooled to 5° C. for 30 min. The product was isolated by filtration, washed by AcOEt, and dried under vacuum to afford Nα-α,β-diaminopropionic acid 1.7 g with 83% yield. TLC (CHCl₃: MeOH: AcOH=15:4:1): Rf=0.19.

Step 2: To a solution of Nα-α,β-diaminopropionic acid (1 g, 5.0 mmol) in a mixture of THF (20 mL) and MeOH (5 mL) was added 2N trimethylsilyldiazomethane in hexane (2.7 mL, 5.4 mmol). The mixture was stirred at room temperature for 48 hours. The solvent was evaporated to get 1.1 g of methyl Nα-α,β-diaminopropionate. Yield 100%. TLC (CHCl₃: MeOH: AcOH=15:4:1): Rf=0.40.

Step 3: A solution of methyl Nα-α,β-diaminopropionate (862 mg, 4.0 mmol) and (6-bromo(2-naphthyl))chlorosulfone (1.2 g, 4.0 mmol) and triethylamine (1.1 mL, 8 mmol) in CH₂Cl₂ (50 mL) was stirred at 0° C. to room temperature for four hours. The solvent was removed and the residue was dissolved in EtOAc (100 mL), washed with sat. NaHCO₃ (30 mL), sat. NaCl (30 mL), 1N HCl (30 mL), sat. NaCl (2×30 mL) dried and evaporated to get 820 mg of product in 43% yield.

Step 4: The compound obtained above was treated with 4N HCl in dioxane at 0° C. for 2 hrs. After removing the solvent, a solution of the residue (200 mg, 0.5 mmol), compound C (120 mg, 0.25 mmol) and triethylamine (0.14 ml) in a mixture of toluene (10 mL) and DMF (1 mL) was refluxed overnight. The reaction mixture was cooled and diluted with EtOAc (20 mL), washed with sat. NaHCO₃ (10 mL), sat. NaCl (10 mL), 1N HCl (10 mL), sat. NaCl (2×10 mL) dried and evaporated to get the crude product, which was treated with TFA (2 mL) at 90° C. for 1 hour. The solvent was evaporated and the mixture was purified by RP-HPLC to afford the title compound. ES-MS: (M+H)⁺=658.1.

### Example 17

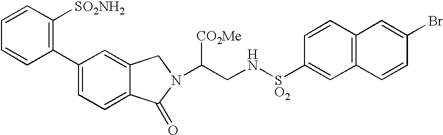

To a solution of the compound obtained in Example 16 in MeOH (5 mL) was added 1N LiOH (0.5 mL), stirred at room temperature for 2 hours. Solvent was removed and the residue was dissolved in EtOAc (10 mL), washed with water, evaporated, and purified with RP-HPLC to afford the title compound. ES-MS: (M+H)⁺=644.0.

### BIOLOGICAL ACTIVITY EXAMPLES

Evaluation of the compounds of this invention is guided by in vitro protease activity assays (see below) and in vivo studies to evaluate antithrombotic efficacy, and effects on hemostasis and hematological parameters.

The compounds of the present invention are dissolved in buffer to give solutions containing concentrations such that assay concentrations range from 0 to 100 μM. In the assays for thrombin, prothrombinase and factor Xa, a synthetic chromogenic substrate is added to a solution containing test compound and the enzyme of interest and the residual catalytic activity of that enzyme is determined spectrophotometrically. The IC₅₀ of a compound is determined from the substrate turnover. The IC₅₀ is the concentration of test compound giving 50% inhibition of the substrate turnover. The compounds of the present invention desirably have an IC₅₀ of less than 500 nM in the factor Xa assay, preferably less than 200 nM, and more preferred compounds have an IC₅₀ of about 100 nM or less in the factor Xa assay. The compounds of the present invention desirably have an IC₅₀ of less than 4.0 μM in the prothrombinase assay, preferably less than 200 nM, and more preferred compounds have an IC₅₀ of about 10 nM or less in the prothrombinase assay. The compounds of the present Invention desirably have an IC₅₀ of greater than 1.0 μM in the thrombin assay, preferably greater than 10.0 μM, and more preferred compounds have an IC₅₀ of greater than 100.0 μM in the thrombin assay.

### Amidolytic Assays for Determining Protease Inhibition Activity

The factor Xa and thrombin assays are performed at room temperature, in 0.02 M Tris.HCl buffer, pH 7.5, containing 0.15 M NaCl. The rates of hydrolysis of the paranitroanilide substrate S-2765 (Chromogenix) for factor Xa, and the substrate Chromozym TH (Boehringer Mannheim) for thrombin following preincubation of the enzyme with inhibitor for 5 minutes at room temperature, and were determined using the Softmax 96-well plate reader (Molecular Devices), monitored at 405 nm to measure the time dependent appearance of p-nitroaniline.

The prothrombinase inhibition assay is performed in a plasma free system with modifications to the method described by Sinha, U. et al., Thromb. Res., 75:427-436 (1994). Specifically, the activity of the prothrombinase complex is determined by measuring the time course of thrombin generation using the p-nitroanilide substrate Chromozym TH. The assay consists of preincubation (5 minutes) of selected compounds to be tested as inhibitors with the complex formed from factor Xa (0.5 nM), factor Va (2 nM), phosphatidyl serine:phosphatidyl choline (25:75, 20 μM) in 20 mM Tris.HCl buffer, pH 7.5, containing 0.15 M NaCl, 5 mM CaCl₂ and 0.1% bovine serum albumin. Aliquots from the complex-inhibitor mixture are added to prothrombin (1 nM) and Chromozym TH (0.1 mM). The rate of substrate cleavage is monitored at 405 nm for two minutes. Eight different concentrations of inhibitor are assayed in duplicate. A standard curve of thrombin generation by an equivalent amount of untreated complex are used for determination of percent inhibition.

### Antithrombotic Efficacy in a Rabbit Model of Venous Thrombosis

A rabbit deep vein thrombosis model as described by Hollenbach, S. et al., Thromb. Haemost. 71:357-362 (1994), is used to determine the in-vivo antithrombotic activity of the test compounds. Rabbits are anesthetized with I.M. injections of Ketamine, Xylazine, and Acepromazine cocktail. A standardized protocol consists of insertion of a thrombogenic cotton thread and copper wire apparatus into the abdominal vena cava of the anesthetized rabbit. A non-occlusive thrombus is allowed to develop in the central venous circulation and inhibition of thrombus growth is used as a measure of the antithrombotic activity of the studied compounds. Test agents or control saline are administered through a marginal ear vein catheter. A femoral vein catheter is used for blood sampling prior to and during steady state infusion of test compound. Initiation of thrombus formation begins immediately after advancement of the cotton thread apparatus into the central venous circulation. Test compounds are administered from time=30 min to time=150 min at which the experiment is terminated. The rabbits are euthanized and the thrombus excised by surgical dissection and characterized by weight and histology. Blood samples are analyzed for changes in hematological and coagulation parameters.

### Effects of Compounds in Rabbit Venous Thrombosis Model

Administration of compounds in the rabbit venous thrombosis model demonstrates antithrombotic efficacy at the higher doses evaluated. There are no significant effects of the compound on the aPTT and PT prolongation with the highest dose (100 μg/kg+2.57 μg/kg/min). Compounds have no significant effects on hematological parameters as compared to saline controls. All measurements are an average of all samples after steady state administration of vehicle or (D)-Arg-Gly-Arg-thiazole. Values are expressed as mean±SD.

Without further description, it is believed that one of ordinary skill in the art can, using the preceding description and the following illustrative examples, make and utilize the compounds of the present invention and practice the claimed methods.

What is claimed is:

1. A compound of the formula I: 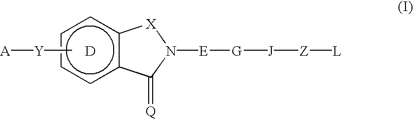
wherein:
A is selected from the group consisting of: (a) C₁-C₆-alkyl; (b) C₃-C₈-cycloalkyl; (c) —N(R,R¹), (R,R¹)N—C(═NR²)—, R¹—C(═NR²)—, (R,R¹)N—C(═NR²)—NR³—, R—C(—NR²)—N(R³)—; (d) phenyl, which is independently substituted with 0-2 R¹ substituents; (e) naphthyl, which is independently substituted with 0-2 R¹ substituents; and (f) a monocyclic or fused bicyclic heterocyclic ring system having from 5 to 10 ring atoms, wherein 1-4 ring atoms of the ring system are selected from N, O and S, and wherein the ring system is optionally substituted with 0-2 R¹ substituents;
R and R¹ are independently selected from the group consisting of H, halo, C₁₋₄alkyl, C₂₋₆alkenyl, C₂₋₆alkynyl, C₃₋₈cycloalkyl, C₀₋₄alkylC₃₋₈cycloalkyl, C₀₋₄alkylphenyl, C₀₋₄alkylnaphthyl, —CN, —NO₂, (CH₂)ₘCON(R²,R³), (CH₂)ₘCO₂R², (CH₂)ₘN(R²,R³), (CH₂)ₘSO₂N(R²,R³), (CH₂)ₘSO₂R², CF₃, OR², N(R²,R³), (R²,R³)N—C(═NR⁴)—, R²—C(—NR⁴)— and a 3-8 membered cyclic system containing from 0-4 heteroatoms selected from N, O and S, wherein from 1-4 hydrogen atoms on the ring system are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂; and wherein R and R¹ taken together may form a ring;
the subscript m is an integer of 0-4;
R² and R³ are independently selected from the group consisting of H, C₁₋₄alkyl, C₂₋₆alkenyl, C₂₋₆alkynyl, C₃₋₈cycloalkyl, C₀₋₄alkylC₃₋₈cycloalkyl, OH, NH₂, OC₁₋₄alkyl, N(C₁₋₄alkyl,C₁₋₄alkyl), C₀₋₄alkylphenyl and C₀₋₄alkylnaphthyl, wherein from 1-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂; and wherein R² and R³ taken together may form a ring;
Y is a member selected from the group consisting of a direct link, —C(═O)—, —CH₂—, —N(R⁴)—CH₂—, —CH₂N(R⁴)—, —N(R⁴)—, —C(═O)—N(R⁴)—, —N(R⁴)—C(═O)—, —C(═NR⁴)—, —C(═NR⁴)—N(R)—, —C(═NR⁴)—CH₂—, —C(═NR⁴)—N(R^{4a})—CH₂—, —S(═O)₂—, —S(═O)—, —O—, —S—, —SO₂—N(R⁴)— and —N(R⁴)—SO₂—;
R⁴ and R^{4a} are independently selected from the group consisting of H, C₁₋₄alkyl, C₂₋₆alkenyl, C₂₋₆alkynyl, C₃₋₈cycloalkyl, C₀₋₄alkylC₃₋₈cycloalkyl, C₀₋₄alkylphenyl and C₀₋₄alkylnaphthyl, wherein from 1-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃cycloalkyl, C₀₋₄alkylC₃₋₈cycloalkyl, —CN and —NO₂;
D is a member selected from the group consisting of (a) phenyl, which is independently substituted with 0-2 R^{1a} substituents; and (b) an aromatic five or six-membered heterocyclic ring having from 1-2 ring hetero atoms selected from oxygen, sulfur and nitrogen atoms, and wherein the ring atoms are optionally substituted with 0-2 R^{1a} substituents; wherein R^{1a} is selected from the group consisting of H, halo, C₁₋₄alkyl, C₂₋₆alkenyl, C₂₋₆alkynyl, C₃₋₈cycloalkyl, C₀₋₄alkylC₃₋₈cycloalkyl, —CN, —NO₂, (CH₂)ₙN(R^{2a},R^{3a}) (CH₂)ₙSO₂N(R^{2a},R^{3a}), (CH₂)ₙSO₂R^{2a}, (CH₂)ₙOR^{2a}, (CH₂)ₙCON(R^{2a},R^{3a}), (CH₂)ₙCO₂R^{2a}, CF₃, and a 5-6 membered aromatic heterocyclic system containing from 1-4 heteroatoms selected from N, O and S, wherein from 0-4 hydrogen atoms on the aromatic heterocyclic system is optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂; the subscript n is an integer of 0-4; and R^{2a} and R^{3a} are independently selected from the group consisting of H, C₁₋₄alkyl, C₂₋₆alkenyl, C₂₋₆alkynyl, C₃₋₈cycloalkyl, C₀₋₄alkylC₃₋₈cycloalkyl, C₀₋₄alkylphenyl and C₀₋₄alkylnaphthyl, wherein from 0-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂;
X is a member selected from the group consisting of —C(R⁵,R^{5a})—C(═O)—, —C(R⁵,R^{5a})—C(═S)—, —C(R⁵,R^{5a})—, —C(R⁵,R^{5a})—C(R⁶,R^{6a}), —C(R⁵,R^{5a})—C(R⁶,R^{6a}) —C(R⁷,R^{7a})—, —C(═O)—, —S(═O)₂—, —C(R⁵)═C(R⁶)—C(═O)—, —C(R⁵)═C(R⁶)—C(═S)—, —C(R⁵)═C(R⁶)—, —O—C(R⁵,R^{5a})—C(═O)—, —S(═O)—, —O—C(R⁵,R^{5a})—C(═S)—, —S—C(R⁵,R^{5a})—C(═O)—, —S—C(R⁵,R^{5a})—C(═S)—, N═C(R⁵)—C(═S)—, —N═C(R⁵)—C(═O)—, —O—C(R⁵,R^{5a})—C(R⁶,R^{6a})—, —N(R⁴)—C(R⁵,R^{5a})—C(R⁶,R^{6a})—, —N(R⁴)—C(R⁵,R^{5a})—, —O—C(R⁵,R^{5a})—, —N═C(R⁵)—; —S(═O)—C(R⁵,R^{5a})—C(R⁶,R^{6a})—, —S(═O)₂—C(R⁵,R^{5a})—C(R⁶,R^{6a})—, —C(═C(R^{5a},R^{5b}))—C(═O)— and —C(═C(R^{5a},R^{5b}))—C(═S)—; wherein the first named atom of each X is directly attached to the D ring, and wherein D, X and the N atom attached to the last chain atom of X collectively form a bicyclic ring structure;
R⁵, R^{5a}, R⁶, R^{6a}, R⁷, and R^{7a} are independently selected from the group consisting of H, C₁₋₄alkyl, C₂₋₆alkenyl, C₂₋₆alkynyl, C₃₋₈cycloalkyl, C₀₋₄alkylC₃₋₈cycloalkyl, NO₂, CF₃, (CH₂)ₙOC₁₋₄alkyl, (CH₂)ₙN(C₁₋₄alkyl, C₁₋₄alkyl), (CH₂)ₙCO₂C₁₋₄alkyl, (CH₂)ₙCON(C₁₋₄alkyl, C₁₋₄alkyl), C₀₋₄alkylphenyl and C₀₋₄alkylnaphthyl, wherein from 0-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂; where two alkyl groups may form a ring and the subscript n has the meaning defined above;
Q is O, or Q and the carbon atom to which it is attached is —CH₂—;
E is a member selected from the group consisting of a direct link, —C(R⁸,R^{8a})—, —C(R⁸,R^{8a})C(R⁹,R^{9a})—, —C(R⁸,R^{8a})C(R⁹,R^{9a})C(R¹⁰,R^{10a})— and —C(═O)—; wherein R⁸, R^{8a}, R⁹, R^{9a}, R¹⁰ and R^{10a} are each independently a member selected from the group consisting of H, C₁₋₄alkyl, C₂₋₆alkenyl, C₂₋₆alkynyl, C₃₋₈cycloalkyl, C₀₋₄alkyl-C₃₋₈cycloalkyl, C₀₋₄alkylphenyl, C₀₋₄alkylnaphthyl, —C₀₋₄alkylCO₂R¹¹, —C₀₋₄alkylC(═O)N(R¹¹,R^{11a}), —C₀₋₄alkylOC₀₋₄alkylR¹¹; —CH₂—CH₂—O—R¹¹, —N(—CH₂—CH₂—O—R¹¹)₂, —C₀₋₄alkylN(R¹¹)C(═O)R¹², —C₀₋₄alkylN(R¹¹)SO₂R¹², C₀₋₄alkylOH, C₀₋₄alkylNR¹¹R^{11a}, C₀₋₄alkylOC₁₋₄alkyl, C₀₋₄alkylN(C₁₋₄alkyl, C₁₋₄alkyl) and a naturally occurring or synthetic amino acid side chain, wherein from 1-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkyl-C₃₋₈cycloalkyl, —CN and —NO₂; R⁸ and R⁹, or R⁹ and R¹⁰, or R⁸ and R^{8a}, or R⁹ and R^{9a} taken together may form a ring;
R¹¹, R^{11a} and R¹² are independently selected from the group consisting of H, C₁₋₄alkyl, C₀₋₄alkylCON(R¹³,R¹⁴), C₀₋₄alkylCOR¹³, C₀₋₄alkylN(R¹³,R¹⁴) and C₀₋₄alkylOR¹³; and wherein R¹¹ and R^{11a}, taken together with N, optionally form a 5-8 membered heterocyclic ring containing 1-4 heteroatoms selected from N, O and S;
R¹³ and R¹⁴ are independently selected from the group consisting of H and C₁₋₄alkyl; or R¹³ and R¹⁴ taken together with N form a 5-8 membered heterocyclic ring containing 1-4 heteroatoms selected from N, O and S;
G is a member selected from the group consisting of a direct link, —O—, —O—C(R¹⁵,R^{15a})—, —N(R¹⁵)—, —N(R¹⁵)—C(R^{15a},R^{15b})—, —S—, —N(R¹⁵)—S(═O)—, —N(R¹⁵)—S(═O)₂—, —S(═O)—N(R¹⁵)—, —S(═O)₂N(R¹⁵)—, —N(R¹⁵)—C(═O)—, —C(═O)—N(R¹⁵)—, —N(R¹⁵)—C(═O)—N(R^{15a})— and a monocyclic aromatic or non-aromatic heterocyclic ring having from 5 to 8 ring atoms, wherein 0-4 ring atoms of the ring system are selected from N, O and S;
R¹⁵, R^{15a} and R^{15b} are independently selected from the group consisting of H, C₁₋₄alkyl, C₂₋₆alkenyl, C₂₋₆alkynyl, C₃₋₈cycloalkyl, C₀₋₄alky1C₃₋₈cycloalkyl, C₀₋₄alkylphenyl, C₀₋₄alkylnaphthyl, C₀₋₄alkylheteroaryl, C₁₋₄alkylCO₂H, C₁₋₄alkylCO₂C₁₋₄alkyl, C₁₋₄alkylCONH₂, C₁₋₄alkylCON(C₁₋₄alkyl, C₁₋₄alkyl), C₂₋₄alkylOH, C₂₋₄alkylNH₂, C₂₋₄alkylOC₁₋₄alkyl and C₂₋₄alkylN(C₁₋₄alkyl, C₁₋₄alkyl), wherein from 1-4 hydrogen atoms on the ring atoms of the phenyl, naphthyl and heteroaryl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂;
J is a member selected from the group consisting of a direct link, —O—, —S—, —N(R¹⁶)—, —N(R¹⁶)—C(═O)—, —C(═O)—N(R¹⁶)—, —N(R¹⁶)—CH₂—, —S(═O)₂, —S(═O)— and —OCH₂—;
R¹⁶ is a member selected from the group consisting of H, C₁₋₄alkyl, C₂₋₆alkenyl, C₂₋₆alkynyl, C₃₋₈cycloalkyl, C₀₋₄alkyl-C₃₋₈cycloalkyl, C₀₋₄alkylphenyl, C₀₋₄alkylnaphthyl, C₀₋₄alkylheterocyclic ring having from 1 to 4 hetero ring atoms selected from the group consisting of N, O and S, —CH₂CO₂C₁₋₄alkyl-, CH₂CO₂H, CH₂CON(C₁₋₄alkyl, C₁₋₄alkyl), CH₂CONH₂, C(═O)C₁₋₄alkyl, SO₂C₁₋₄alkyl, CH₂CO₂—C₁₋₄alkylphenyl and CH₂CO₂C₁₋₄alkylnaphthyl;
Z is a member selected from the group consisting of (a) phenyl, which is independently substituted with 0-2 R^{1b} substituents; (b) naphthyl, which is independently substituted with 0-2 R^{1b} substituents; and (c) a monocyclic or fused bicyclic heterocyclic ring system having from 5 to 10 ring atoms, wherein 1-4 ring atoms of the ring system are selected from N, O and S, and wherein the ring system is optionally substituted from 0-2 R^{1b} substituents;
R^{1b} is selected from the group consisting of H, halo, C₁₋₄alkyl, C₂₋₆alkenyl, C₂₋₆alkynl, C₃₋₈cycloalkyl, C₀₋₄alkylC; C₃₋₈cycloalkyl, —CN, —NO₂, N(R^{2b},R^{3b}), SO₂N(R^{2b},R^{3b}), SO₂R^{2b}, CO₂N(R^{2b},R^{3b}), CO₂R^{2b}, CF₃, OR^{2b}, O—CH₂—CH₂—OR^{2b}, O—CH₂—CH₂—N(R^{2b},R^{3b}), O—CH₂—CON(R^{2b},R^{3b}), O—CH₂—CH₂—N(R^{2b},R^{3b}), O—CH₂—COOR^{2b}, N(R^{2b})—CH₂—CH₂—OR^{3b}, N(—CH₂—CH₂—O^{2b})₂, N(R^{2b})—C(═O)R^{3b}, N(R^{2b})—SO₂—R^{3b} and a 5-6 membered aromatic heterocyclic system containing from 1-4 heteroatoms selected from N, O and S, wherein from 1-4 hydrogen atoms on the aromatic heterocyclic system are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂;
R^{2b} and R^{3b} are independently selected from the group consisting of H, C₁₋₄alkyl, C₂₋₆alkenyl, C₂₋₆alkynyl, C₃₋₈cycloalkyl, C₀₋₄alkylC₃₋₈cycloalkyl, C₀₋₄alkylphenyl and C₀₋₄alkylnaphthyl, wherein from 1-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN and —NO₂;
L is selected from the group consisting of H, —CN, C(═O)N(R¹⁷,R^{17a}), (CH₂)ₙN(R¹⁷, R^{17a}R), C(═NR¹⁷)N(R^{17a},R^{17b}), OR¹⁷, —NR¹⁷C(═NR^{17a})N(R^{17b},R^{17c}) and NR¹⁷C(═NR^{17a})—R^{17b};
R¹⁷, R^{17a}, R^{17b}, and R^{17c} are independently selected from the group consisting of H, —OR¹⁸, —NR¹⁸R^{18a}, C₁₋₄alkyl, C₀₋₄alkylphenyl, C₀₋₄alkylnaphthyl, COOC₁₋₄alkyl, COO—C₀₋₄alkylphenyl and COO—C₀₋₄alkylnaphthyl, wherein from 1-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN, and —NO₂;
R¹⁸ and R^{18a} are independently selected from the group consisting of H, C₁₋₄alkyl, C₂₋₆alkenyl, C₂₋₆alkynyl, C₃₋₈cycloalkyl, C₀₋₄alkylC₃₋₈cycloalkyl, C₀₋₄alkylphenyl and C₀₋₄alkylnaphthyl, wherein from 1-4 hydrogen atoms on the ring atoms of the phenyl and naphthyl moieties are optionally independently replaced with a member selected from the group consisting of halo, C₁₋₄alkyl, C₂₋₆alkenyl, C₂₋₆alkynyl, C₃₋₈cycloalkyl, C₀₋₄alkylC₃₋₈cycloalkyl, —CN, and —NO₂;
and all pharmaceutically acceptable isomers, salts, hydrates, solvates and prodrug derivatives thereof.

2. The compound according to claim 1 with a general formula I, wherein:
R and R¹ are independently selected from the group consisting of H, halo, C₁₋₄alkyl, —CN, —NO₂, (CH₂)ₘCON(R²,R³), (CH₂)ₘCO₂R², (CH₂)ₘN(R²,R³), SO₂N(R²,R³), SO₂R², CF₃, OR², N(R²,R³), (R²,R³)N—C(═NR⁴)—, R²—C(═NR⁴)— and a 3-8 membered cyclic system containing from 0-4 heteroatoms selected from N, O and S;
m is an integer of 0-4;
R² and R³ are independently selected from the group consisting of H, C₁₋₄alkyl, C₂₋₆alkenyl, C₂₋₆alkynyl, C₃₋₈cycloalkyl, C₀₋₄alkylC₃₋₈cycloalkyl, OH, NH₂, OC₁₋₄alkyl, N(C₁₋₄alkyl,C₁₋₄alkyl), C₀₋₄alkylphenyl and C₀₋₄alkylnaphthyl;
Y is a member selected from the group consisting of a direct link, —C(═O)—, —CH₂—, —N(R⁴)—CH₂—, —CH₂N(R⁴)—, —N(R⁴)—, —C(═NR⁴)—, —C(═NR⁴)—N(R)—, —C(═NR⁴)—CH₂—, —C(═NR⁴)_N(R^{4a})—CH₂—, —O— and —S—;
R⁴ and R^{4a} are independently selected from the group consisting of H, C₁₋₄alkyl, C₂₋₆alkenyl, C₂₋₆alkynyl, C₃₋₈cycloalkyl, C₀₋₄alkylC₃₋₈cycloalkyl, C₀₋₄alkylphenyl and C₀₋₄alkylnaphthyl;
D is a member selected from the group consisting of
(a) phenyl, which is independently substituted with 0-2 R^{1a} substituents; and
(b) an aromatic five or six-membered heterocyclic ring having from 1-2 ring hetero atoms selected from oxygen, sulfur and nitrogen atoms, and wherein the ring atoms are optionally substituted with 0-2 R^{1a} substituents;
wherein each R^{1a} is a member selected from the group consisting of H, halo, C₁₋₄alkyl, —CN, —NO₂, (CH₂)ₙNR^{2a}R^{3a}, (CH₂)ₙSO₂NR^{2a}R^{3a}, (CH₂)ₙSO₂R^{2a}, (CH₂)ₙOR^{2a}, (CH₂)ₙCONR^{2a}R^{3a}, (CH₂)ₙCO₂R^{3a} and CF₃,
n is an integer of 0-4;
R^{2a} and R^{3a} are members independently selected from the group consisting of H, C₁₋₄alkyl, C₀₋₄alkylphenyl and C₀₋₄alkylnaphthyl;
X is a member selected from the group consisting of —C(R⁵,R^{5a})—C(═O), —C(R⁵,R^{5a})—, —C(R⁵,R^{5a})—C(R⁶,R^{6a}) —, —C(R⁵,R^{5a})—C(R⁶,R^{6a})—C(R⁷,R^{7a})—, —C(═O)—, —S(═O)₂—, —C(R⁵)═C(R⁶)—, —S(═O)—, —C)—C(R⁵,R^{5a})—C(R⁶,R^{6a})—, —N(R⁴)—C(R⁵,R^{5a})—C(R⁶,R^{6a})—, —N(R⁴)—C(R⁵,R^{5a})—, —O—C(R⁵,R^{5a})— and —N═C(R⁵)—;
R⁵, R^{5a}, R⁶, R^{6a}, R⁷, and R^{7a} are independently selected from the group consisting of H, C₁₋₄alkyl, NO₂, CF₃, (CH₂)ₙOC₁₋₄alkyl, (CH₂)ₙN(C₁₋₄alkyl, C₁₋₄alkyl), (CH₂)ₙCO₂C₁₋₄alkyl, (CH₂)ₙCON(C₁₋₄alkyl, C₁₋₄alkyl), C₀₋₄alkylphenyl and C₀₋₄alkylnaphthyl; two alkyl taken together may form a ring, and n is as defined before;
Q is a member selected from the group consisting of ═H₂ and ═O;
E is a member selected from the group consisting of a direct link, —C(R⁸,R^{8a})—, —C(R⁸,R^{8a})C(R⁹,R^{9a})—, —C(R⁸,R^{8a})C(R⁹,R^{9a})C(R¹⁰,R^{10a})— and —C(═O)—;
wherein R⁸, R^{8a}, R⁹, R^{9a}, R¹⁰ and R^{10a} are independently members selected from the group consisting of H, C₁₋₄alkyl, C₀₋₄alkylphenyl, C₀₋₄alkylnaphthyl, —C₀₋₄alkylCO₂R¹¹, —C₀₋₄alkylC(═O)N(R¹¹,R^{11a}), —C₀₋₄alkylOC₀₋₄alkylR¹¹, —CH₂—CH₂—O—R¹¹, —N(—CH₂—CH₂—O—R¹¹)₂, —C₀₋₄alkylN(R¹¹)C(═O)R¹², —C₀₋₄alkylN(R¹¹)SO₂R¹², C₀₋₄alkylOH, C₀₋₄alkylNH₂, C₀₋₄alkylOC₁₋₄alkyl, C₀₋₄alkylN(C₁₋₄alkyl, C₁₋₄alkyl) and a naturally occurring or synthetic amino acid side chain;
wherein R¹¹, R^{11a} and R¹² are independently a member selected from the group consisting of H, C₁₋₄alkyl, C₀₋₄alkylCON(R¹³,R¹⁴), C₀₋₄alkylCOR¹³, C₀₋₄alkylN(R¹³,R¹⁴) and C₀₋₄alkylOR¹³; or R¹¹ and R^{11a} taken together with N may form a 5-8 membered ring containing 1-4 heteroatoms selected from N, O and S;
R¹³ and R¹⁴ are independently a member selected from the group consisting of H and C₁₋₄alkyl; or R¹³ and R¹⁴ taken together with N may form a 5-8 membered heterocyclic ring containing 1-4 heteroatoms selected from N, O and S;
G is a member selected from the group consisting of a direct link, —O—, —O—C(R¹⁵,R^{15a})—, —N(R¹⁵)—, N(R¹⁵)—C(R^{15a},R^{15b})—, —S—, —N(R¹⁵)—S(═O)—, —N(R¹⁵)—S(═O)₂—, —S(═O)—N(R¹⁵)—, —S(═O)₂N(R¹⁵)—, —N(R¹⁵)—C(═O)—, —C(═O)—N(R¹⁵)—, —N(R¹⁵)—C(═O)—N(R^{15a})— and a monocyclic aromatic or non-aromatic ring having from 5 to 8 ring atoms, wherein 0-4 ring atoms of the ring system are selected from N, O and S;
R¹⁵, R^{15a} and R^{15b} are independently a member selected from the group consisting of H, C₁₋₄alkyl, C₀₋₄alkylphenyl, C₀₋₄alkylnaphthyl, C₁₋₄alkylCO₂H, C₁₋₄alkylCO₂C₁₋₄alkyl, C₁₋₄alkylCONH₂, C₁₋₄alkylCON(C₁₋₄alkyl, C₁₋₄alkyl), C₂₋₄alkylOH, C₂₋₄alkylNH₂, C₂₋₄alkylOC₁₋₄alkyl and C₂₋₄alkylN(C₁₋₄alkyl, C₁₋₄alkyl);
J is a member selected from the group consisting of a direct link, —O—, —S—, —N(R¹⁶)—, —N(R¹⁶)—C(═O)—, —C(═O)—N(R¹⁶)—, —N(R¹⁶)—CH₂—, —S(═O)₂—, —S(═O)— and —OCH₂—;
R¹⁶ is a member selected from the group consisting of H, C₁₋₄alkyl, C₀₋₄alkylphenyl, C₀₋₄alkylnaphthyl, CH₂CO₂C₁₋₄alkyl, CH₂CO₂H, CH₂CON(C₁₋₄alkyl, C₁₋₄alkyl), CH₂CONH₂, C(═O)C₁₋₄alkyl, SO₂C₁₋₄alkyl, CH₂CO₂—C₁₋₄alkylphenyl and CH₂CO₂C₁₋₄alkylnaphthyl;
Z is a member selected from the group consisting of (a) phenyl, which is independently substituted with 0-2 R^{1b} substituents; (b) naphthyl, which is independently substituted with 0-2 R^{1b} substituents; and (c) a monocyclic or fused bicyclic heterocyclic ring system having from 5 to 10 ring atoms, wherein 1-4 ring atoms of the ring system are selected from N, O and S, and wherein the ring system is optionally substituted from 0-2 R^{1b} substituents;
R^{1b} is a member selected from the group consisting of H, halo, C₁₋₄alkyl, —CN, —NO₂, N(R^{2b}R^{3b}), SO₂N(R^{2b},R^{3b}), SO₂R^{2b}, CO₂N(R^{2b},R^{3b}), CO₂R^{2b}, CF₃, OR^{2b}, O—CH₂—CH₂—OR^{2b}, O—CH₂—CH₂—N(R^{2b},R^{3b}), O—CH₂—CON(R^{2b},R^{3b}), O—CH₂—CO₂R^{2b}, N(R^{2b})—CH₂—CH₂—OR^{3b}, N(—CH₂—CH₂—OR^{2b})₂, N(R^{2b})—C(═O)R^{3b} and N(R^{2b})—SO₂—R^{3b};
R^{2b} and R^{3b} are independently selected from the group consisting of H, C₁₋₄alkyl, C₁₋₄alkylphenyl and C₁₋₄alkylnaphthyl;
L is a member selected from the group consisting of H, —CN, C(═O)N(R¹⁷,R^{17a}), (CH₂)ₙN(R¹⁷,R^{17a}), C(═NR¹⁷)N(R^{17a},R^{17b}), OR¹⁷, —NR¹⁷C(═NR^{17a})N(R^{17b},R^{17c}) and NR¹⁷C(═NR^{17a})—R^{17b};
R¹⁷, R^{17a}, R^{17b}, and R^{17c} are independently selected from H, —OR¹⁸, —N(R¹⁸,R^{18),} C₁₋₄alkyl, C₀₋₄alkylphenyyl and C₀₋₄alkylnaphthyl;
R¹⁸ and R^{18a} are independently selected from H, C₁₋₄alkyl, C₀₋₄alkylphenyl and C₀₋₄alkylnaphthyl;
and all pharmaceutically acceptable isomers, salts, hydrates, solvates and prodrug derivatives thereof.

3. The compound according to claim 2, wherein:
A is a member selected from the group consisting of: 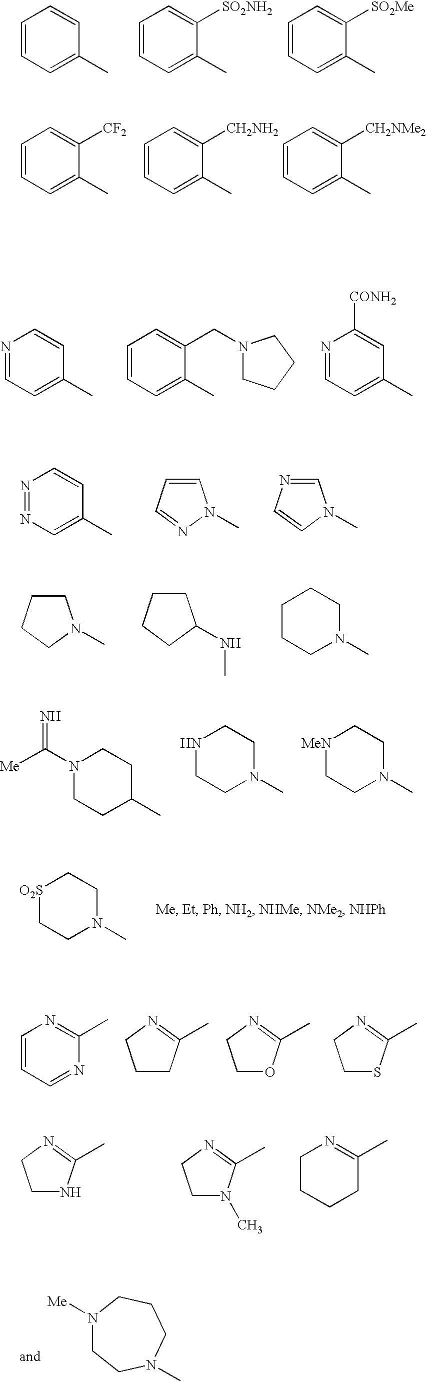
Y is a member selected from the group consisting of a direct link, —C(═O)—, —CH₂—, —NH—CH₂—, —NMe-CH₂—, —NH—, —NMe-, —C(═NH)—, —C(═NMe)-, —O— and —S—;
the portion provided as 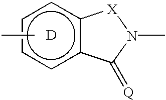
is a member selected from the group consisting of 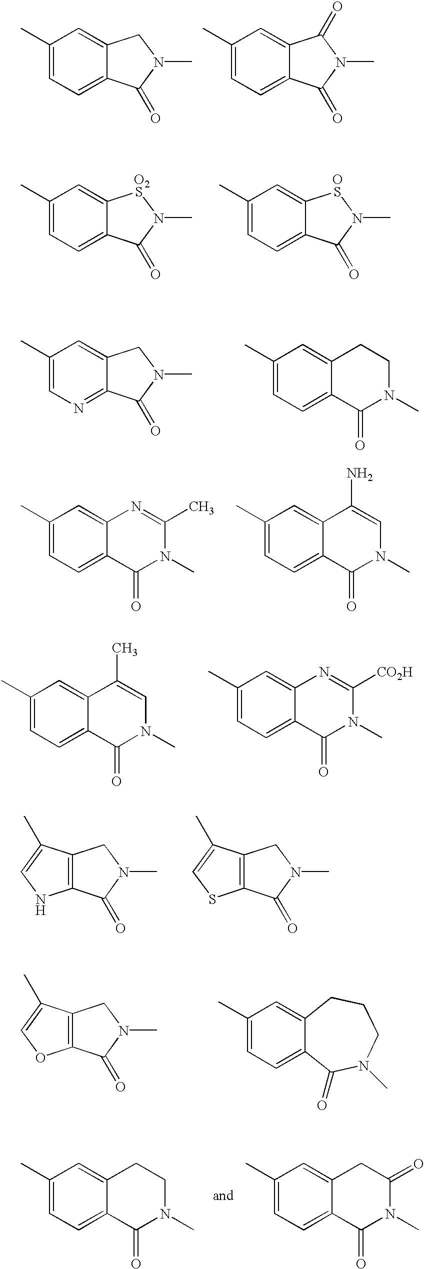
E is a member selected from the group consisting of a direct link, —CH(R⁸)—, —CH(R⁸)CH₂—, —CH(R⁸)CH₂CH₂— and —C(═O)—;
R⁸ is a member selected from the group consisting of H, OH, NH₂, NHAc, Me Et, Ph, Bn, cyclohexyl, CO₂H, CO₂Me, CONH₂, CONMe₂, CONHMe, CH₂CO₂H, CH₂CO₂Me, CH₂CONH₂, CH₂CONMe₂, CH₂CONHMe, 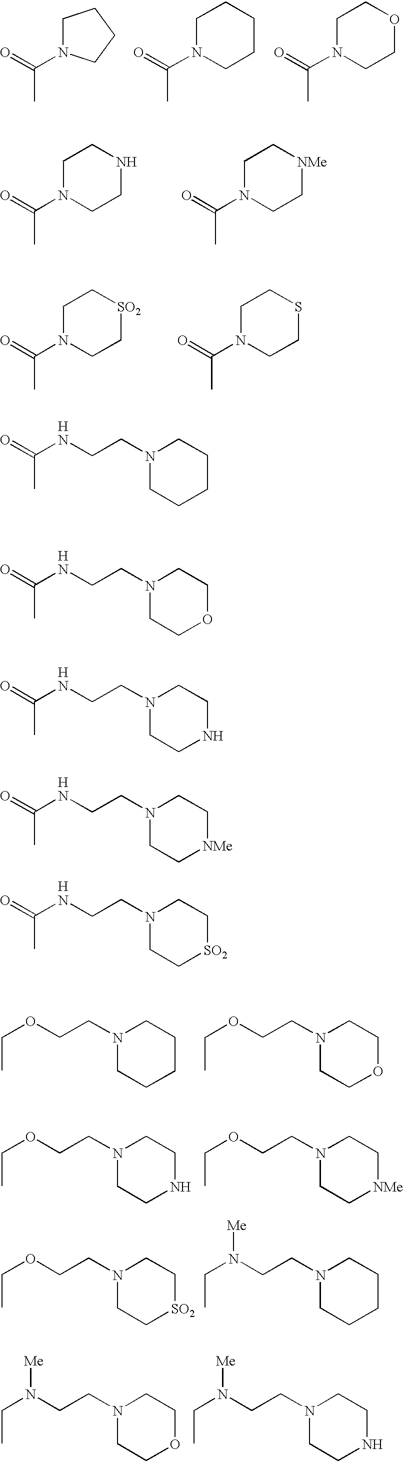
G is a member selected from the group consisting of a direct link, —O—, —N(R¹⁵)—, —S—, —N(R¹⁵)—S(═O)—, —N(R¹⁵)—S(═O)₂—, —N(R¹⁵)—C(═O)—, —C(═O)—N(R¹⁵)—, —N(R¹⁵)—C(═O)—N(R^{15a})—, 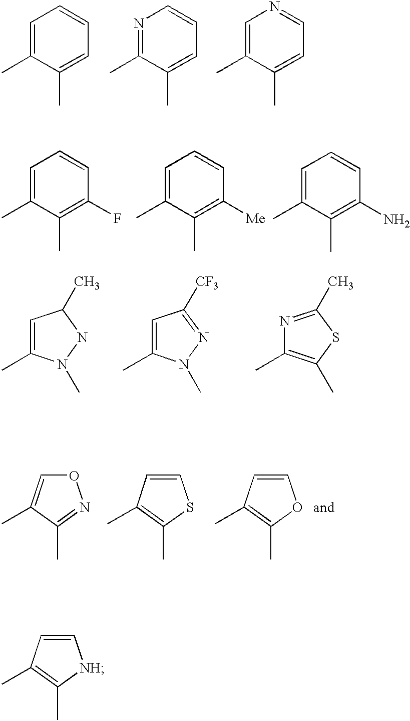
R¹⁵ and R^{15a} are independently selected from H, Me, Et, Bn, CH₂CO₂H, CH₂CO₂Me, CH₂CONH₂ and CH₂CONMe₂;
J is a member selected from the group consisting of a direct link, —O—, —S—, —NH—, —NMe-, —NH—C(═O)—, —C(═O)—NH—; —NMe-C(═O)— and —C(═O)—NMe-;
Z-L together is a member selected from the group consisting of: 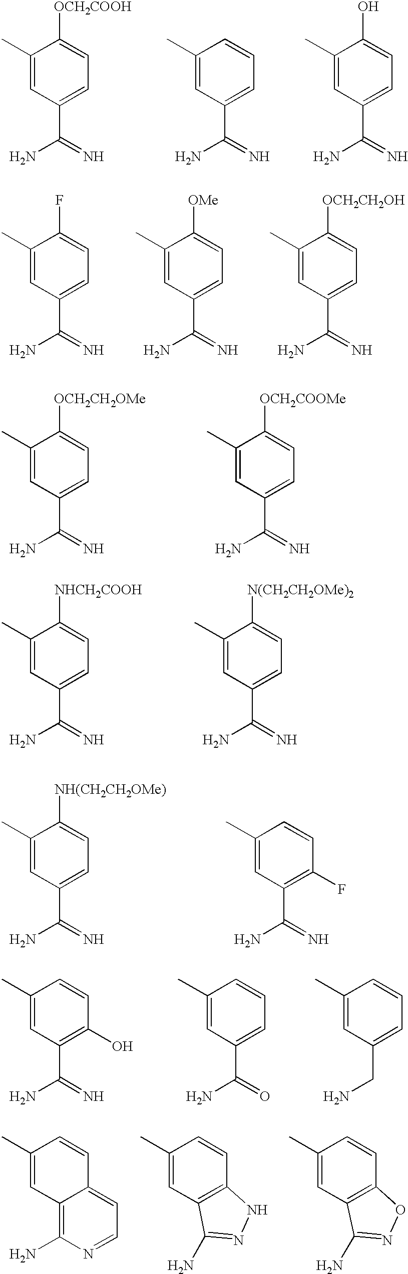
and all pharmaceutically acceptable isomers, salts, hydrates, solvates and prodrug derivatives thereof.

4. A compound having a formula selected from the group consisting of:
(a) compounds of formulae Ia, Ib, Ic and Id, 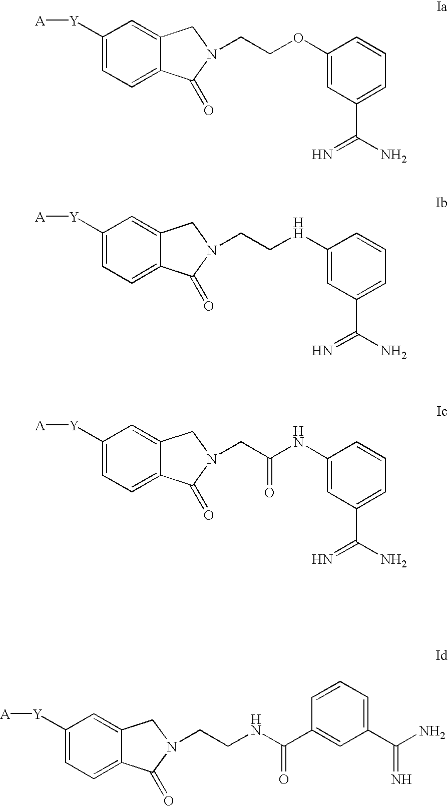
wherein:
A is a member selected from the group consisting of: 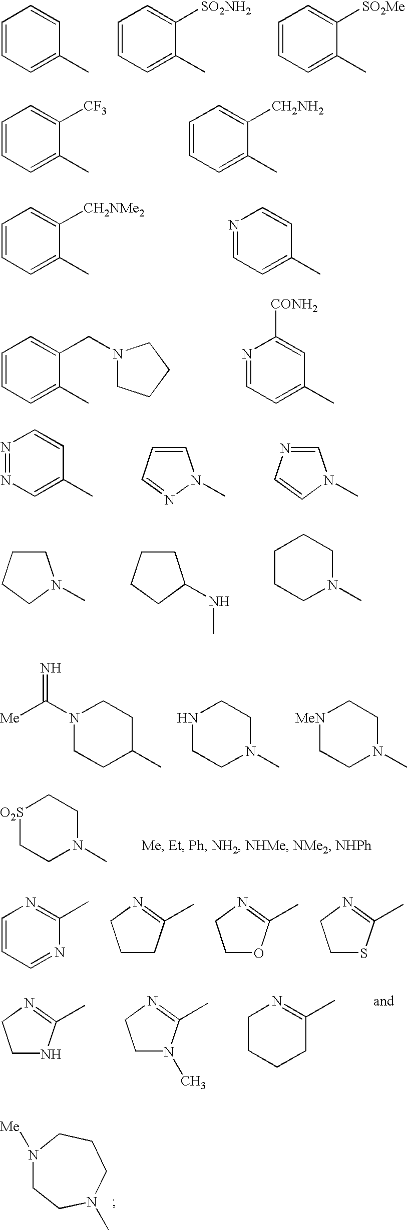
and Y is a member selected from the group consisting of:
a direct link, —O—, —S—, —SO₂—, —SO—, —C(═O)—, —CH₂—, —NH—CH₂—, —NMe-CH₂—, —CH₂N(R⁴)—, —C(═NH)—, —C(═NH)—CH₂—, —C(═NMe)-CH₂—, —C(═NMe)-, —NH—, —NMe-, —C(═O)—NH—, —NH—C(═O)—, —C(═NH)—NH—, —C(═NH)—NMe- and —C(═NMe)—NH—;
(b) compounds of formulae IIa, IIb, IIc and IId, 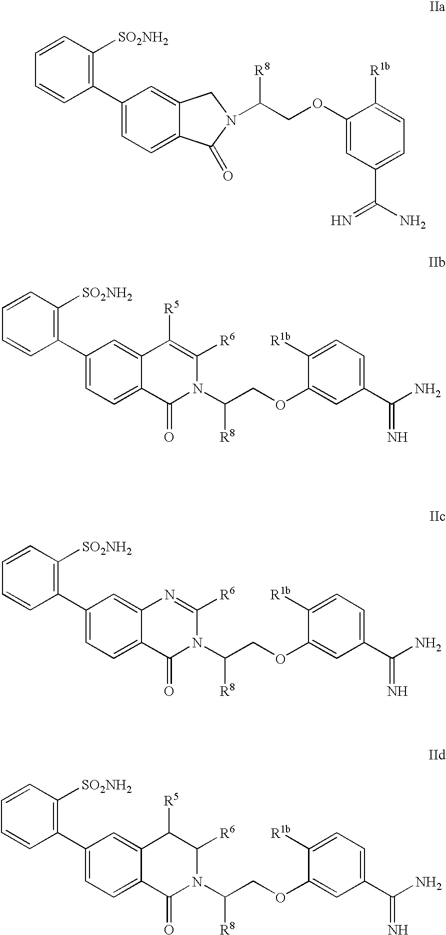
wherein:
R^{1b} is a member selected from the group consisting of:
H, F, Cl, Br I, Me, OH, OMe, OPh, OBn, NH₂, OCH₂CH₂NH₂, OCH₂CH₂OH, CONH₂, NO₂, SO₂Me, SO₂NH₂, CN, CH₂OH, CH₂NH₂, CO₂H, CO₂Me, CH₂CO₂H, CH₂CO₂Me, CONMe₂ and CH₂CONMe₂;
R⁵ and R⁶ are independently a member selected from the group consisting of:
H, Me, Et, CF₃, Ph, Bn, CO₂H, CO₂Me, CONH₂, CONMe₂, NH₂, OH, CH₂NH₂, CH₂OH, CH₂CO₂H, CH₂CO₂Me, CH₂CONH₂ and CH₂CONMe₂; and
R⁸ is a member selected from the group consisting of:
H, Me, Et, Ph, Bn, cyclohexyl, CH₂cyclohexyl, CH₂NH₂, CH₂OH, CONMe₂, CONH₂, CH₂CONMe₂, CH₂CONH₂, CO₂H, CO₂Me, CH₂CO₂H, CH₂CO₂Me, naphthyl, CH₂naphthyl, CONMe₂ and CH₂CONMe₂;
(c) compounds of formulae IIIa, IIIb, IIIc and IIId, 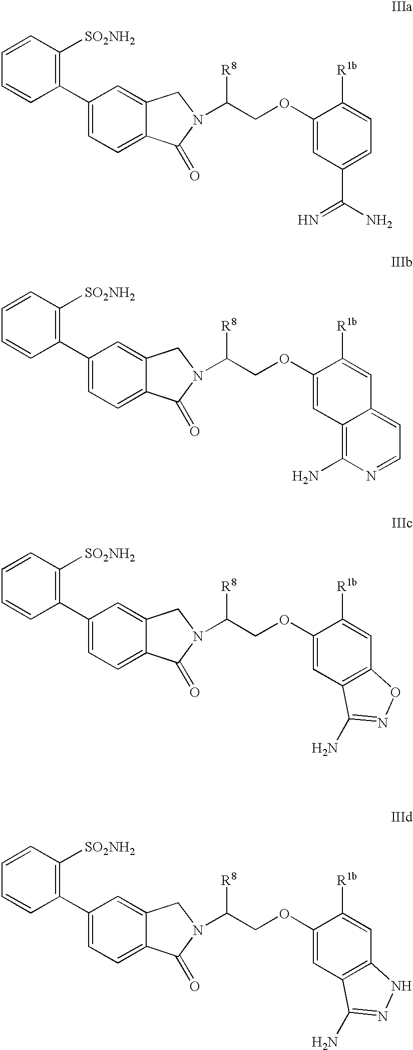
wherein:
R^{1b} is a member selected from the group consisting of:
H, F, Cl, Br I, Me, OH, OMe, OPh, OBn, NH₂, OCH₂CH₂NH₂, OCH₂CH₂OH, CONH₂, NO₂, SO₂Me, SO₂NH₂, CN, CH₂OH, CH₂NH₂, CO₂H, CO₂Me, CH₂CO₂H, CH₂CO₂Me, CONMe₂ and CH₂CONMe₂; and
R⁸ is a member selected from the group consisting of:
H, Me, Et, Ph, Bn, cyclohexyl, CH₂cyclohexyl, CH₂NH₂, CH₂OH, CONMe₂, CONH₂, CH₂CONMe₂, CH₂CONH₂, CO₂H, CO₂Me, CH₂CO₂H, CH₂CO₂Me, aryl, CH₂aryl, CONMe₂ and CH₂CONMe₂;
(d) compounds of formulae IVa, IVb, IVc, IVd and IVe, 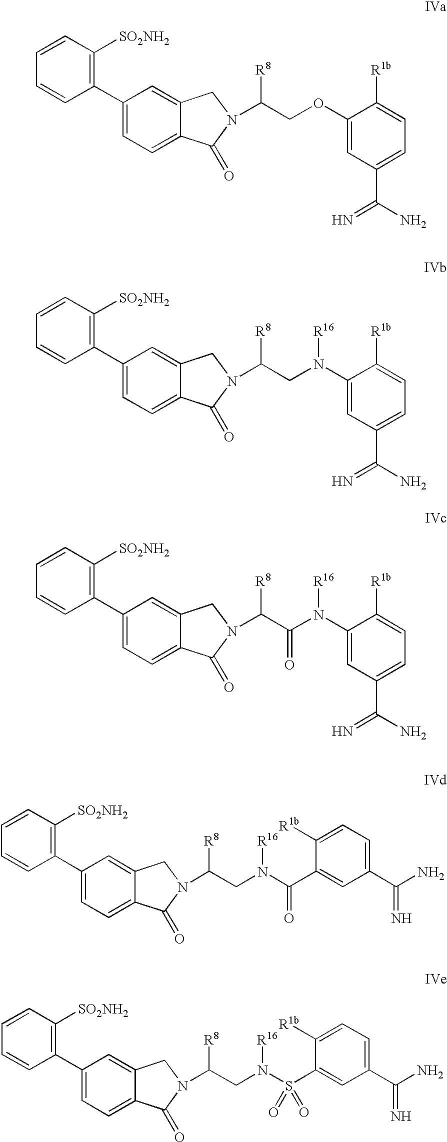
wherein:
R^{1b} is a member selected from the group consisting of:
H, F, Cl, Br, I, Me, OH, OMe, OPh, OBn, NH₂, OCH₂CH₂NH₂, OCH₂CH₂OH, CONH₂, NO₂, SO₂Me, SO₂NH₂, CN, CH₂OH, CH₂NH₂, CO₂H, CO₂Me, CH₂CO₂H, CH₂CO₂Me, CONMe₂ and CH₂CONMe₂;
R⁸ is a member selected from the group consisting of:
H, Me, Et, Ph, Bn, cyclohexyl, CH₂cyclohexyl, CH₂NH₂, CH₂OH, CONMe₂, CONH₂, CH₂CONMe₂, CH₂CONH₂, CO₂H, CO₂Me, CH₂CO₂H, CH₂CO₂Me, CONMe₂ and CH₂CONMe₂; and
R¹⁶ is a member selected from the group consisting of:
H, Me, Et, Ph, and Bn;
(e) compounds of formulae Va, Vb, Vc, Vd, Ve and Vf, 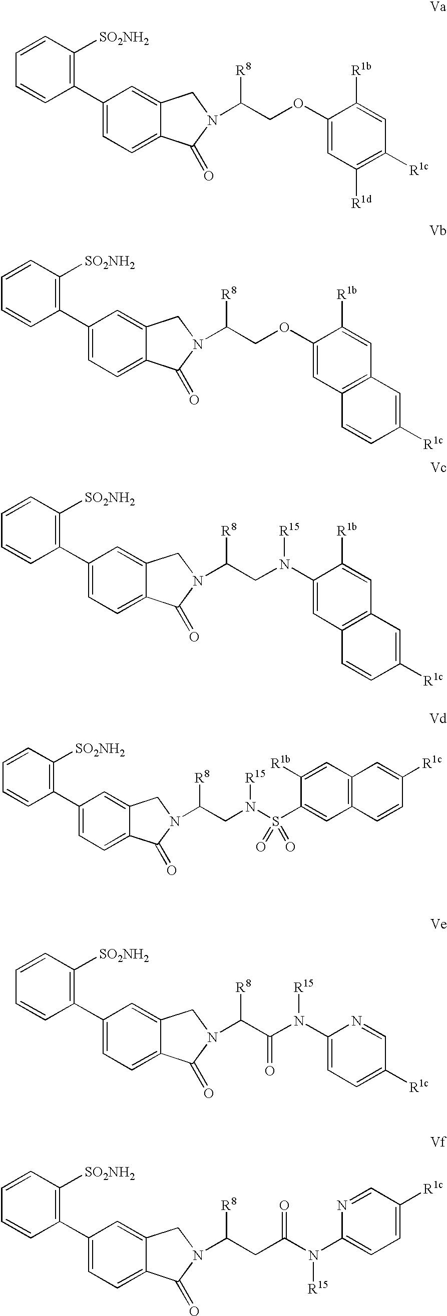
wherein:
R^{1b}, R^{1c} and R^{1d} are individually a member selected from the group consisting of:
H, F, Cl, Br, I, Me, OH, OMe, OPh, OBn, NH₂, OCH₂CH₂NH₂, OCH₂CH₂OH, CONH₂, NO₂, SO₂Me, SO₂NH₂, CN, CH₂OH, CH₂NH₂, CO₂H, CO₂Me, CH₂CO₂H, CH₂CO₂Me, CONMe₂ and CH₂CONMe₂;
R⁸ is a member selected from the group consisting of:
H, Me, Et, Ph, Bn, cyclohexyl, CH₂cyclohexyl, CH₂NH₂, CH₂OH, CONMe₂, CONH₂, CH₂CONMe₂, CH₂CONH₁₂, CO₂H, CO₂Me, CH₂CO₂H, CH₂CO₂Me, naphthyl, CH₂naphthyl, CONMe₂ and CH₂CONMe₂;
R¹⁵ is a member selected from the group consisting of:
H, Me, Et, Ph, and Bn;
(f) compounds of formulae VIa, VIb, VIc, VId, VIe, VIf, VIg, VIh, VIi, VIj, VIk and VIl, 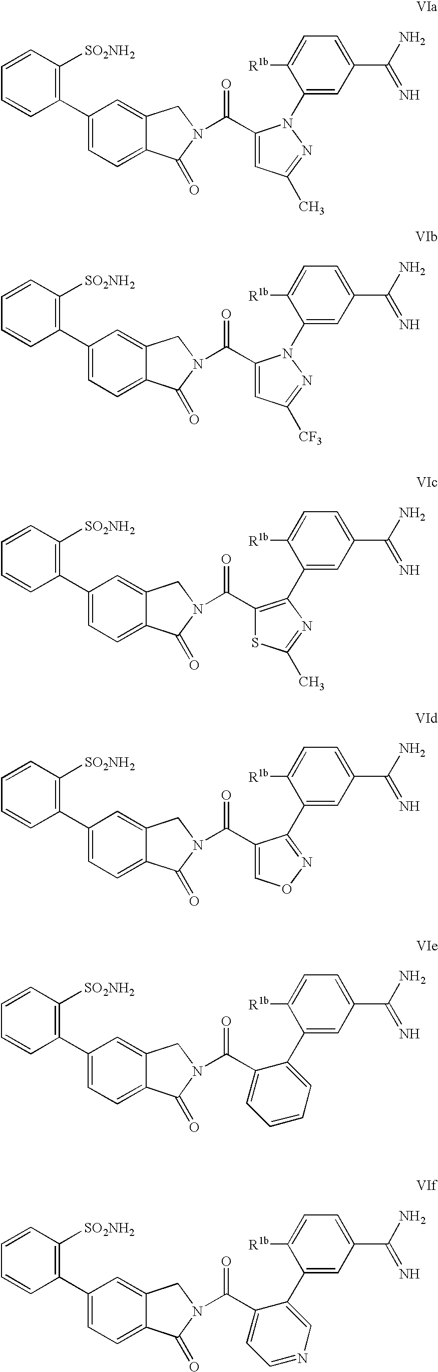
wherein:
R^{1b} is a member selected from the group consisting of:
H, F, Cl, Br, I, Me, OH, OMe, OPh, OBn, NH₂, OCH₂CH₂NH₂, OCH₂CH₂OH, CONH₂, NO₂, SO₂Me, SO₂NH₂, CN, CH₂OH, CH₂NH₂, CO₂H, CO₂Me, CH₂CO₂H, CH₂CO₂Me, CONMe₂ and CH₂CONMe₂; and
(g) compounds of formula VIIa, VIIb and VIIc, 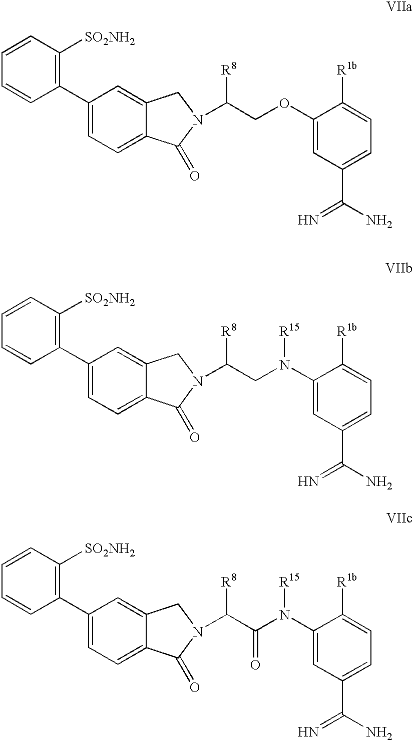
wherein:
R^{1b} is a member selected from the group consisting of:
H, OH, NH₂, NO₂, F, SO₂Me, CN, CONH₂ and SO₂NH₂;
R⁸ is a member selected from the group consisting of:
H, Me, Et, Ph, Bn, CO₂H and CO₂Me; and
R¹⁵ is a member selected from the group consisting of:
H, Me, Et and Bn.

5. A pharmaceutical composition for preventing or treating a condition in a mammal characterized by undesired thrombosis comprising a pharmaceutically acceptable carrier and a compound of claim 1.

6. A method for preventing or treating a condition in a mammal characterized by undesired thrombosis comprising the step of administering to said mammal a therapeutically effective amount of a compound of claim 1.

7. The method of claim 6, wherein the condition is selected from the group consisting of:
acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, and conditions requiring the fitting of prosthetic devices.

8. A method for inhibiting the coagulation of biological samples comprising the step of administering a compound of claim 1.
